(19) United States
(12) Patent Application Publication
(10) Pub. No.: US 2017/0218382 A1
(43) Pub. Date: Aug. 3, 2017

(54) Secretion Signal Peptide, And Protein Secretory Production And Cell Surface Display Using Said Secretion Signal Peptide

(71) Applicant: NATIONAL UNIVERSITY CORPORATION KOBE UNIVERSITY, Hyogo (JP)

(72) Inventors: Akihiko KONDO, Hyogo (JP); Tomohisa HASUNUMA, Hyogo (JP); Kentaro INOKUMA, Hyogo (JP)

(73) Assignee: National University Corporation Kobe University, Hyogo (JP)

(21) Appl. No.: 15/328,766

(22) PCT Filed: Jul. 30, 2015
(86) PCT No.: PCT/JP2015/071608

§ 371 Date: Jan. 24, 2017

(30) Foreign Application Priority Data
Jul. 31, 2014 (JP) 2014-156796

(51) Int. Cl. C12N 15/81; C12N 9/42; C12P 21/00; C07K 14/395

(52) U.S. Cl. CPC C12N 15/81; C07K 14/395; C12N 9/2445; C12Y 302/01021; C12N 9/2434; C12P 21/00

(57) ABSTRACT

An expression vector is disclosed which contains a promoter DNA; a DNA encoding a peptide having a defined amino acid sequence and having secretion signal activity; and a DNA encoding an intended protein or a cloning site for insertion of the DNA encoding an intended protein. An expression vector is also disclosed which contains a promoter DNA; a DNA encoding any peptide having a defined amino acid sequence and having secretion signal activity; a DNA encoding an intended protein or a cloning site for insertion of the DNA encoding an intended protein; and a DNA encoding an anchor domain. The peptide having secretion signal activity allows for secretory production and cell surface display of a protein with high activity, in yeast. According to the present invention, a secretion signal peptide is provided which stably has higher secretion activity ability It is also an object of the present invention to provide a secretion signal peptide that stably has higher secretion ability than that of a conventionally used secretion signal peptide in secretory production and cell surface display of a protein.

### TECHNICAL FIELD

The present invention relates to a secretion signal peptide, and a protein secretory production and cell surface display using the secretion signal peptide.

### BACKGROUND ART

In recent years, introduction of a technique for manufacturing ethanol from lignocellulosic biomass, which is a renewable resource, has been in demand from a viewpoint of realizing a low-carbon recycling-oriented society. In order to promote widespread use of this technique, it is urgently necessary to construct a low-cost simultaneous saccharification-fermentation process in which multiple steps of converting plant materials into ethanol are integrated to improve the efficiency.

For the purpose of constructing this process, yeasts to which a saccharification ability is imparted by enzymes such as cellulase and hemicellulase being displayed on the cell surface are produced. An example of a cell surface display technique is a method using a GPI anchor protein, which is a cell surface-localized protein. Such a cell surface display technique uses an expression cassette containing a promoter, a secretion signal peptide, a gene for surface display, and a gene for a GPI anchor protein, for example.

There are various reports about the GPI anchor protein and the promoter (Patent Documents 1 to 4, and Non-Patent Documents 1 to 5).

The secretion signal peptide is a peptide present at the N-terminus of a protein to be localized in a cell membrane or cell wall, or to be secreted extracellularly. It is reported that an expression efficiency and expression success rate in a protein secretory expression system can be improved by substituting a secretion signal peptide for an intended protein with a highly efficient secretion signal peptide. Therefore, an improvement in efficiency of the secretion signal peptide is an important element in an improvement of efficiency of a protein cell surface display system and a protein secretory production system.

An example of a typical secretion signal peptide derived from Saccharomyces cerevisiae used in the existing highly efficient secretory protein expression system for a yeast is a secretion signal peptide (MFα prepro) derived from an α-factor (Non-Patent Document 6). A secretion signal peptide derived from Saccharomyces cerevisiae that is considered to have secretion ability higher than that of the secretion signal peptide derived from an a-factor is also reported (Patent Document 5).

On the other hand, it is known that the secretion ability of the secretion signal peptide varies greatly depending on a combination with a promoter and a protein to be connected to the upstream and the downstream of the secretion signal peptide. Moreover, it is currently difficult to predict the stability of the secretion ability from the sequence of the secretion signal peptide. Therefore, even when a secretion signal sequence that can exert high secretion ability in combination with a specific promoter and a specific protein is used, there may be many cases where the secretion ability decreases significantly when the secretion signal sequence is used in combination with another promoter and another protein.

For example, the secretion ability of a secretion signal peptide that is considered to be highly efficient in Patent Document 5 is evaluated using, as an index, only the activity of the secretion signal peptide used in combination of a single promoter (HSP1 promoter) and a single protein (luciferase).

Furthermore, the secretion ability of a secretion signal peptide that is considered to be highly efficient in Patent Document 5 is evaluated using, as an index, only the activity of the secretion signal peptide when an intended protein is secreted extracellularly (into the culture supernatant).

### RELATED ART DOCUMENTS

### Patent Documents

Patent Document 1: Japanese Laid-Open Patent Publication No. 2011-160727

Patent Document 2: Japanese Laid-Open Patent Publication No. 2008-86310

Patent Document 3: Japanese Laid-Open Patent Publication No. 2007-189909

Patent Document 4: Japanese Laid-Open Patent Publication No. 2005-245335

Patent Document 5: Japanese Laid-Open Patent Publication No. 2007-167062

Patent Document 6: Japanese Laid-Open Patent Publication No. 2011-30563

### Non-Patent Documents

Non-Patent Document 1: Biotechnol. Lett., 2010, vol. 32, pp. 1131-1136

Non-Patent Document 2: Mol. Microbiol., 2004, vol. 52, pp. 1413-1425

Non-Patent Document 3: Appl. Environmen. Microbiol., 1997, vol. 63, pp. 615-620

Non-Patent Document 4: Biotechnol. Lett., 2010, vol. 32, pp. 255-260

Non-Patent Document 5: J. Bacteriol., 1997, vol. 179, pp. 1513-1520

Non-Patent Document 6: Protein Eng., 1996, vol. 9, pp. 1055-1061

Non-Patent Document 7: Appl. Microbiol. Biotechnol., 2006, vol. 72, pp. 1136-1143

Non-Patent Document 8: Nature Methods, 2009, vol. 6, pp. 343-345

Non-Patent Document 9: FEMS Yeast Res., 2014, vol. 14, pp. 399-411

Non-Patent Document 10: Biotechnol. Biofuels, 2014, vol. 7, p. 8

Non-Patent Document 11: Enzyme Microb. Technol., 2012, vol. 50, pp. 343-347

Non-Patent Document 12: J Biochem., 2012, vol. 145, pp. 701-708

### SUMMARY OF INVENTION

### Problem to be Solved by the Invention

It is an object of the present invention to provide a secretion signal peptide that has a higher secretion ability than that of a conventionally used secretion signal peptide in secretory production and cell surface display of a protein.

It is also an object of the present invention to provide a secretion signal peptide that stably has higher secretion ability than that of a conventionally used secretion signal peptide when used in combination with a plurality of different promoters and proteins.

### Means for Solving the Problems

The present invention provides an expression vector (the expression vector is also referred to as a secretion-type expression vector) comprising:

(i) a promoter DNA;

(ii) a DNA encoding any peptide selected from the group consisting of:
(a) a peptide that has the amino acid sequence of SEQ ID No. 2;
(b) a peptide that has an amino acid sequence having at least 70% sequence identity to the amino acid sequence of SEQ ID No. 2, and has secretion signal activity;
(c) a peptide that has an amino acid sequence obtained by substitution, deletion, or addition of one or several amino acid residues in the amino acid sequence of SEQ ID No. 2, and has secretion signal activity;
(d) a peptide that is encoded by a base sequence having at least 70% sequence identity to the base sequence of SEQ ID No. 1, and has secretion signal activity; and
(e) a peptide that is encoded by a base sequence that can hybridize with a complementary strand of a DNA having the base sequence of SEQ ID No. 1, and has secretion signal activity; and

(iii) a DNA encoding an intended protein or a cloning site for insertion of the DNA encoding an intended protein.

In an embodiment, in the secretion-type expression vector, the promoter is an SED1 promoter.

In an embodiment, in the secretion-type expression vector, the item (iii) is a DNA encoding an intended protein.

The present invention provides a transformed yeast into which the secretion-type expression vector has been introduced.

The present invention provides a method for producing a yeast performing protein secretory production, the method comprising:

obtaining a transformed yeast by introducing an expression cassette into a yeast, the expression cassette comprising a promoter DNA; a DNA encoding any peptide selected from the group consisting of (a) a peptide that has the amino acid sequence of SEQ ID No. 2, (b) a peptide that has an amino acid sequence having at least 70% sequence identity to the amino acid sequence of SEQ ID No. 2, and has secretion signal activity, (c) a peptide that has an amino acid sequence obtained by substitution, deletion, or addition of one or several amino acid residues in the amino acid sequence of SEQ ID No. 2, and has secretion signal activity, (d) a peptide that is encoded by a base sequence having at least 70% sequence identity to the base sequence of SEQ ID No. 1, and has secretion signal activity and (e) a peptide that is encoded by a base sequence that can hybridize with a complementary strand of a DNA having the base sequence of SEQ ID No. 1, and has secretion signal activity; and a DNA encoding an intended protein.

The present invention provides a method for performing protein secretory production in a yeast, the method comprising culturing the transformed yeast or a yeast produced using the method.

The present invention provides an expression vector (the expression vector is also referred to as a surface display-type expression vector) comprising:

(i) a promoter DNA;

(ii) a DNA encoding any peptide selected from the group consisting of
(a) a peptide that has the amino acid sequence of SEQ ID No. 2;
(b) a peptide that has an amino acid sequence having at least 70% sequence identity to the amino acid sequence of SEQ ID No. 2, and has secretion signal activity;
(c) a peptide that has an amino acid sequence obtained by substitution, deletion, or addition of one or several amino acid residues in the amino acid sequence of SEQ ID No. 2, and has secretion signal activity;
(d) a peptide that is encoded by a base sequence having at least 70% sequence identity to the base sequence of SEQ ID No. 1, and has secretion signal activity; and
(e) a peptide that is encoded by a base sequence that can hybridize with a complementary strand of a DNA having the base sequence of SEQ ID No. 1, and has secretion signal activity;

(iii) a DNA encoding an intended protein or a cloning site for insertion of the DNA encoding an intended protein; and

(iv) a DNA encoding an anchor domain.

In an embodiment, in the surface display-type expression vector, wherein the promoter is an SED1 promoter.

In an embodiment, in the surface display-type expression vector, the anchor domain is an SED1 anchor domain.

In an embodiment, in the surface display-type expression vector, the item (iii) is a DNA encoding an intended protein.

The present invention provides a transformed yeast into which the surface display-type expression vector has been introduced.

The present invention provides a method for producing a yeast displaying a protein on a surface, the method comprising:

obtaining a transformed yeast by introducing an expression cassette into a yeast, the expression cassette comprising a promoter DNA; a DNA encoding any peptide selected from the group consisting of (a) a peptide that has the amino acid sequence of SEQ ID No. 2, (b) a peptide that has an amino acid sequence having at least 70% sequence identity to the amino acid sequence of SEQ ID No. 2, and has secretion signal activity, (c) a peptide that has an amino acid sequence obtained by substitution, deletion, or addition of one or several amino acid residues in the amino acid sequence of SEQ ID No. 2, and has secretion signal activity, (d) a peptide that is encoded by a base sequence having at least 70% sequence identity to the base sequence of SEQ ID No. 1, and has secretion signal activity and (e) a peptide that is encoded by a base sequence that can hybridize with a complementary strand of a DNA having the base sequence of SEQ ID No. 1, and has secretion signal activity; a DNA encoding an intended protein; and a DNA encoding an anchor domain.

### Effects of the Invention

According to the present invention, a protein can be secreted or displayed on a cell surface with high activity. The secretion signal peptide of the present invention can be preferably used for both secretion and cell surface display of a protein. Furthermore, the secretion signal peptide of the present invention can stably impart high secretion ability when used in combination with various promoters and various protein genes.

### BRIEF DESCRIPTION OF DRAWINGS

FIG. 1 is a graph illustrating the time course of β-glucosidase activity of cells for various transformed surface display yeasts obtained by using expression cassettes X1 to X4, during culture.

FIG. 2 is a graph illustrating the time course of β-glucosidase activity in culture media for various transformed secretion yeasts obtained by using expression cassettes X5 to X8, during culture.

FIG. 3 is a graph illustrating endoglucanase activity of cells for various transformed surface display yeasts obtained by using expression cassettes X9 to X11, after 48 hours of culture.

FIG. 4 is a graph illustrating the time course of β-glucosidase activity of cells for various transformed surface display yeasts obtained by using expression cassettes X12 to X17, during culture.

FIG. 5 is a graph illustrating the time course of β-glucosidase activity in culture media for various transformed secretion yeasts obtained by using expression cassettes X18 and X19, during culture.

FIG. 6 is a graph illustrating relative values of GFP fluorescence intensity in culture media for various transformed secretion yeasts obtained by using expression cassettes X20 to X22, after 24 hours of culture.

### MODE FOR CARRYING OUT THE INVENTION

Hereinafter, the present invention will be described in detail.

Secretion Signal Peptide

A secretion signal peptide is a peptide that is generally bound to the N-terminus of a protein to be localized in a cell membrane or cell wall, or to be secreted outward from a cell membrane. In general, the secretion signal peptide is removed by being degraded by a signal peptidase during a process in which a secretory protein is secreted from the inside of a cell through a cell membrane out of the cell. For example, the secretion signal peptide is linked to the N-terminus of a protein (secretory protein) that originally has this secretion signal peptide or a heterologous protein in a cell in which the proteins is to be expressed, so that it can function to allow the secretory protein or the heterologous protein to be secreted out of the cell.

Herein, “functioning as a secretion signal peptide on the expression of an intended protein in a yeast” is also referred to as “secretion signal activity”.

According to the present invention, any peptide selected from the group consisting of (a) to (e) below is provided as a secretion signal peptide:

(a) a peptide that has the amino acid sequence of SEQ ID No. 2;

(b) a peptide that has an amino acid sequence having at least 70% sequence identity to an amino acid sequence of SEQ ID No. 2 and has secretion signal activity;

(c) a peptide that has an amino acid sequence obtained by substitution, deletion, or addition of one or several amino acid residues in the amino acid sequence of SEQ ID No. 2 and has secretion signal activity;

(d) a peptide tide that is encoded by a base sequence having at least 70% sequence identity to the base sequence of SEQ ID No. 1 and has secretion signal activity; and

(e) a peptide tide that is encoded by a base sequence that can hybridize with a complementary strand of a DNA having the base sequence of SEQ ID No. 1 and has secretion signal activity.

Furthermore, according to the present invention, a polynucleotide including a DNA encoding any peptide (also referred to as merely “secretion signal peptide of the present invention” herein) selected from the group consisting of (a) to (e) above is also provided. For example, a DNA encoding the secretion signal peptide of the present invention has the base sequence of SEQ ID No. 1, which encodes the amino acid sequence of SEQ ID No. 2.

The amino acid sequence of SEQ ID No. 2 and the base sequence (SEQ ID No. 1) encoding this amino acid sequence are derived from the secretion signal peptide of SED1 which is a main cell surface-localized protein in a stationary phase of yeast Saccharomyces cerevisiae, but the source is not limited thereto. SED1 is induced by stress, and is considered to contribute to maintenance of the integrity of a cell wall. The gene (Sed1) for SED1 can be obtained using a method commonly used by a person skilled in the art based on sequence information registered in GenBank (GenBank accession number NM_001180385; NCBI Gene ID: 851649), for example.

Here, the protein or peptide described herein may be a protein or peptide that has an amino acid sequence obtained by deletion, substitution, or addition of one or several amino acids in the disclosed amino acid sequence and that substantially has a desired function or effect in the present invention, and the polynucleotide may include a polynucleotide encoding such a protein or peptide. Any one of or a combination of two or more of the amino acid mutations (e.g., deletion, substitution, and addition) may be introduced into the disclosed amino acid sequence. The total number of mutations is one or several, but is not particularly limited as long as the protein or peptide substantially has a desired function or effect, and can be dependent on the size of the protein or peptide. Examples of the total number of mutations include one or more and ten or less, one or more and five or less, one or more and four or less, one or more and three or less, and one or more and two or less, but is not limited thereto. The total number of mutations can be within a range that satisfies the sequence identity, which will be described below, for example. With respect to examples of amino acid substitution, any substitution may be used as long as a function or effect is substantially retained. Conservative substitution may be used, for example. Examples of the conservative substitution include substitution within the following groups (i.e., between the amino acids in parentheses): (glycine, alanine), (valine, isoleucine, leucine), (aspartic acid, glutamic acid), (asparagine, glutamine), (serine, threonine), (lysine, arginine), and (phenylalanine, tyrosine).

In another embodiment, the protein or peptide described herein may be a protein or peptide that has an amino acid sequence having 70% or more sequence identity to the disclosed amino acid sequence, and substantially has a desired function or effect in the present invention, for example, and the polynucleotide may include a polynucleotide encoding such a protein or peptide. Moreover, the sequence identity of the amino acid sequence can be 74% or more, 78% or more, 80% or more, 85% or more, 90% or more, 92% or more, 95% or more, 98% or more, or 99% or more.

Sequence identity or similarity as used herein refers to, as is known in the art, the relationship between two or more proteins or two or more polynucleotides that is determined by comparing the sequences. The “identity” of sequences means the degree of sequence invariance between protein sequences or polynucleotide sequences as determined by an alignment between the protein sequences or polynucleotide sequences or in some cases by an alignment between a series of partial sequences. The “similarity” means the degree of correlation between protein sequences or polynucleotide sequences as determined by an alignment between the protein sequences or polynucleotide sequences or in some cases by an alignment between a series of partial sequences. More specifically, the similarity is determined based on the sequence identity and conservativeness (substitution that maintains a particular amino acid in a sequence or physicochemical properties of a sequence). It should be noted that the similarity is called “Similarity” in sequence homology search results of BLAST, which will be described later. It is preferable that the method for determining the identity and similarity is a method that is designed so that the alignment between sequences to be compared becomes the longest. Methods for determining the identity and similarity are offered as programs available to the public. For example, the BLAST (Basic Local Alignment Search Tool) program by Altschul et al. (e.g., Altschul et al., J. Mol. Biol., 1990, 215: 403-410; Altschyl et al., Nucleic Acids Res., 1997, 25: 3389-3402) can be used for determination. Although there is no particular limitation on the conditions in the case where software such as BLAST is used, it is preferable to use default values.

In yet another embodiment, the protein or peptide described herein may be encoded by a DNA that hybridizes with a DNA having a base sequence complementary to a DNA having the disclosed base sequence, and the polynucleotide may include such a hybridizing DNA. It is preferable that hybridization with the DNA having a base sequence complementary to a DNA having the disclosed base sequence is performed in stringent conditions.

The stringent conditions refer to conditions in which a so-called specific hybrid is formed while nonspecific hybrid is not formed, for example. An example thereof is conditions in which a complementary strand of a nucleic acid whose base sequence has high identity, that is, a DNA that has a base sequence having 70% or more, 75% or more, 78% or more, 80% or more, 85% or more, 90% or more, 92% or more, 95% or more, 98% or more, or 99% or more identity to the disclosed base sequence, for example, hybridizes, while a complementary strand of a nucleic acid having homology lower than that does not hybridize. More specifically, such conditions include a sodium salt concentration of 15 mM to 750 mM, 50 mM to 750 mM, or 300 mM to 750 mM, for example, a temperature of 25° C. to 70° C., 50° C. to 70° C., or 55° C. to 65° C., for example, and a formamide concentration of 0% to 50%, 20% to 50%, or 35% to 45%, for example. Furthermore, in the stringent conditions, washing conditions for a filter after the hybridization include a sodium salt concentration of 15 mM to 600 mM, 50 mM to 600 mM, or 300 mM to 600 mM, for example, and a temperature of 50° C. to 70° C., 55° C. to 70° C., or 60° C. to 65° C., for example. An example of a DNA that hybridizes in stringent conditions is a DNA that can be obtained by performing hybridization at 65° C. in the presence of 0.7 to 1.0 M NaCl using a filter on which a DNA is immobilized and then washing the filter in an SSC solution having 0.1 to 2 times concentration (SSC solution of one time concentration has a composition containing 150 mM NaCl and 15 mM sodium citrate) at 65° C. Hybridization can be performed using a well-known method such as a method described in Sambrook et al., Molecular Cloning, A Laboratory Manual, 3rd Ed., Cold Spring Harbor Laboratory (2001), for example. The higher the temperature is and the lower the salt concentration is, the higher the stringency is, thus making it possible to isolate a polynucleotide having higher homology (sequence identity).

In yet another embodiment, examples of the polynucleotide described herein include polynucleotides that have a base sequence having 70% or more, 75% or more, 78% or more, 80% or more, 85% or more, 90% or more, 92% or more, 95% or more, 98% or more, or 99% or more identity to the disclosed base sequence, and substantially have a desired function or effect.

Substitution based on degeneracy of a genetic code can also be performed on a base sequence encoding a predetermined amino acid sequence (e.g., the amino acid sequence of SEQ ID No. 2) to substitute at least one base in the base sequence encoding a predetermined amino acid sequence with a different base without changing the amino acid sequence of a protein. In yet another embodiment, a DNA encoding the secretion signal peptide of the present invention also encompasses a DNA having a base sequence changed by substitution based on degeneracy of a genetic code.

A DNA encoding the secretion signal peptide of the present invention or a polynucleotide containing this DNA can be obtained as a nucleic acid fragment by PCR from a nucleic acid derived from a DNA extracted from a given yeast (e.g., Saccharomyces cerevisiae), various cDNA libraries, or various genome DNA libraries as a template using primers designed based on the base sequence of SEQ ID No. 1, for example. A DNA encoding the secretion signal peptide of the present invention or a polynucleotide containing this DNA can be obtained as a nucleic acid fragment by hybridization on a nucleic acid derived from the aforementioned libraries using a probe designed based on the base sequence of SEQ ID No. 1. A DNA encoding the secretion signal peptide of the present invention or a polynucleotide containing this DNA may be synthesized as a nucleic acid fragment by use of various methods for synthesizing a nucleic acid sequence that are known in the art, such as chemical synthesis.

In the aforementioned various embodiments in which the polynucleotides are described, a DNA encoding the secretion signal peptide of the present invention can be obtained by modifying a DNA having the base sequence of SEQ ID No. 1 by commonly used mutagenesis, site-specific mutagenesis, molecular evolution using error-prone PCR, or the like, for example. Examples of such methods include known methods, such as the Kunkel method or the gapped duplex method, or equivalent methods thereof. For example, a mutation is introduced using a mutation introducing kit (e.g., Mutant-K (manufactured by TAKARA) or Mutant-G (manufactured by TAKARA)) or the like that uses site-directed mutagenesis or using a LA PCR in vitro Mutagenesis Kit from TAKARA.

As described in detail below, expression cassettes such as a secretion cassette for secretory production of an intended protein and a surface display cassette for surface display of an intended protein can be produced using a DNA encoding the secretion signal peptide of the present invention or a polynucleotide containing this DNA.

DNA Encoding Anchor Domain

The surface display cassette contains a DNA encoding an anchor domain.

A cell surface-localized protein or a cell membrane binding region thereof can be used as the anchor domain. The “cell surface-localized protein” refers to a protein that is immobilized on or attaches or adheres to a cell surface and is localized on the cell surface. Lipid-modified proteins are known as the cell surface-localized protein and are immobilized on a cell membrane through a covalent bond between the lipid and membrane components.

A typical example of the cell surface-localized protein is a GPI (glycosyl phosphatidyl inositol: glycolipid having, as a basic structure, ethanolamine phosphate-6 mannose α-1,2 mannose α-1,6 mannose α-1,4 glucosamine α-1,6 inositol phospholipid) anchor protein. The GPI anchor protein has glycolipid GPI at its C-terminus, and is bound to the cell membrane surface through a covalent bond between the GPI and PI (phosphatidyl inositol) in the cell membrane.

GPI is bound to the C-terminus of the GPI anchor protein as follows. After transcription and translation, the GPI anchor protein is secreted into the lumen of the endoplasmic reticulum due to an action of a secretion signal present on the N-terminal side. A region that is recognized when a GPI anchor is bound to a GPI anchor protein, called a GPI anchor attachment signal, is present at or near the C-terminus of the GPI anchor protein. In the lumen of the endoplasmic reticulum and the Golgi body, the GPI anchor attachment signal region is cleaved, and GPI is bound to a newly generated C-terminus.

The protein to which the GPI is bound is transferred through secretion vesicles to the cell membrane, and is immobilized on the cell membrane through a covalent bond of the GPI with the PI in the cell membrane. Then, the GPI anchor is cleaved by phosphatidylinositol-dependent phospholipase C (PI-PLC), and the protein is incorporated into the cell wall so as to be displayed on the cell surface in the state of being immobilized on the cell wall.

In the present invention, a polynucleotide that encodes the entirety of a GPI anchor protein, which is a cell surface-localized protein, or a region including a GPI anchor attachment signal region, which is a cell membrane binding region thereof, can be used. The cell membrane binding region (GPI anchor attachment signal region) is generally a region on the C-terminal side of the cell surface-localized protein. It is sufficient that the cell membrane-binding region includes the GPI anchor attachment signal region, and the cell membrane-binding region may further include any other moiety of the GPI anchor protein as long as the enzyme activity of the fusion protein is not inhibited.

It is sufficient that the GPI anchor protein is a protein that functions in a yeast cell. Examples of the GPI anchor protein include a-agglutinin or a-agglutinin (AGα1, AGA1), TIP1, FLO1, SED1, CWP1, and CWP2. It is preferable to use SED1 or the cell membrane binding region (GPI anchor attachment signal region) thereof. The base sequence of the anchor protein-coding region of Sed1 is listed as SEQ ID No. 4, and the amino acid sequence of the encoded protein is listed as SEQ ID No. 5 (proviso that SEQ ID Nos. 4 and 5 do not include the initiation codon and methionine encoded by the initiation codon). The cell membrane binding region (GPI anchor attachment signal region) of SED1 is a region from position 109 to position 337 of SEQ ID No. 5, for example. The SED1 anchor domain may have the entire amino acid sequence of SEQ ID No. 5, or may have a partial sequence thereof (e.g., sequence including the amino acid sequence from position 109 and to 337 of SEQ ID No. 5) as long as the anchor function is not impaired.

The above-described sequence identity, hybridization conditions, and the like are applied to the anchor domain and a DNA encoding the anchor domain.

A DNA encoding the anchor domain (e.g., the cell surface-localized protein or the cell membrane binding region thereof) can be obtained as a nucleic acid fragment by PCR from a nucleic acid derived from a DNA extracted from microorganisms including the anchor domain, various cDNA libraries, or various genome DNA libraries as a template using primers designed based on known sequence information. Such a polynucleotide can be obtained as a nucleic acid fragment by hybridization on a nucleic acid derived from the aforementioned libraries using a probe designed based on known sequence information. A nucleic acid fragment excised from an existing vector containing the above-mentioned polynucleotide can also be used. The polynucleotide may be synthesized as a nucleic acid fragment using various methods for synthesizing a nucleic acid sequence that are known in the art, such as chemical synthesis.

Promoter DNA

It is sufficient that a promoter DNA has promoter activity. The “promoter activity” refers to activity that allows a transcription factor to be bound to a promoter region so as to induce transcription. The promoter DNA can be excised from cells, phages, and the like harboring a desired promoter region, using a restriction enzyme. A DNA fragment of a promoter region can be obtained by amplifying a desired promoter region by PCR using primers provided with a restriction enzyme recognition site or a site overlapping a cloning vector as necessary. Furthermore, a desired promoter DNA may be chemically synthesized based on base sequence information of an already known promoter region.

Any promoter can be used as the promoter DNA contained in the polynucleotide of the present invention as long as the promoter has promoter activity in a yeast. The promoter DNA may be originally contained in a gene to be expressed or derived from a different gene. The promoter DNA may be a promoter of a gene encoding a cell surface-localized protein to be used as the anchor domain. Examples thereof include an SED1 promoter, a TDH3 promoter, a PGK1 promoter, a CWP2 promoter, and a TDH1 promoter. These promoters have respective base sequences of SEQ ID Nos. 3, 12, 13, 18, and 21. As mentioned above, base sequences mutated by deletion, addition, or substitution of one or two or more (e.g., several) nucleotides, for example, in these base sequences may also be used as long as a desired function can be served.

Intended Protein

There is no particular limitation on the type or source of intended protein.

Examples of the type of intended protein include an enzyme, an antibody, a ligand, and a fluorescent protein. Examples of the enzyme include a cellulose-degrading enzyme, a starch-degrading enzyme, a glycogen-degrading enzyme, a xylan-degrading enzyme, a chitin-degrading enzyme, and a lipid-degrading enzyme, and more specifically include endoglucanase, cellobiohydrolase, β-glucosidase, amylase (e.g., glucoamylase and α-amylase), and lipase.

A DNA encoding the intended protein preferably has a cDNA sequence including no introns.

A DNA encoding the intended protein may have a sequence encoding the entire intended protein, or a sequence encoding a partial region thereof as long as the activity of the intended protein is exhibited. Furthermore, as mentioned above, a DNA encoding the intended protein may have a base sequence mutated by deletion, addition, or substitution of one or two or more (e.g., several) nucleotides, for example, in a base sequence encoding a naturally occurring protein, or a base sequence encoding a protein having an amino acid sequence mutated by deletion, addition, or substitution of one or two or more (e.g., several) amino acids, for example, as long as the activity of the intended protein is exhibited.

A DNA (gene) encoding the intended protein can be obtained as a nucleic acid fragment by PCR from a nucleic acid derived from a DNA extracted from microorganisms containing or producing the intended protein, various cDNA libraries, or various genome DNA libraries as a template using primers designed based on known sequence information. Such a polynucleotide can be obtained as a nucleic acid fragment by hybridization on a nucleic acid derived from the aforementioned libraries using a probe designed based on known sequence information. A nucleic acid fragment (which may be an expression cassette form) excised from an existing vector containing a DNA encoding the intended protein can also be used. Such a gene can also be obtained by artificial synthesis based on a sequence optimized considering the codon usage of a host as necessary.

Hereinafter, a description will be given using a cellulose-degrading enzyme as an example of the intended protein.

The cellulose-degrading enzyme refers to any enzyme that can cleave a β,4-glycosidic linkage. The cellulose-degrading enzyme can be derived from any bacteria that produce a cellulose-hydrolyzing enzyme. Typical examples of the bacteria that produce a cellulose-hydrolyzing enzyme include microorganisms belonging to the genus Aspergillus (e.g., Aspergillus aculeatus, Aspergillus niger, and Aspergillus oryzae), the genus Trichoderma (e.g., Trichoderma reesen), the genus Clostridium (e.g., Clostridium thermocellum), the genus Cellulomonas (e.g., Cellulomonas fimi and Cellulomonas uda), the genus Pseudomonas (e.g., Pseudomonas fluorescence), and the like.

Hereinafter, endoglucanase, cellobiohydrolase, and β-glucosidase will be described as typical cellulose-degrading enzymes, but the cellulose-degrading enzyme is not limited thereto.

Endoglucanase is an enzyme that is usually referred to as cellulase, and it intramolecularly cleaves cellulose to generate glucose, cellobiose, and cello-oligosaccharide (“intramolecular cellulose cleaving”). There are five types of endoglucanase and they are referred to as endoglucanase I, endoglucanase II, endoglucanase III, endoglucanase IV, and endoglucanase V, respectively. They are different from each other in terms of amino acid sequences, but commonly have an intramolecular cellulose cleaving action. For example, endoglucanase (especially endoglucanase EGII (Patent Document 6, for example)) derived from Trichoderma reesei can be used, but there is no limitation thereto.

Cellobiohydrolase degrades cellulose from either the reducing terminus or the non-reducing terminus thereof so as to liberate cellobiose (“cellulose molecule terminal cleaving”). There are two types of cellobiohydrolase and they are referred to as cellobiohydrolase I and cellobiohydrolase II, respectively. They are different from each other in terms of amino acid sequences, but commonly have a cellulose molecule terminal cleaving action. For example, cellobiohydrolase (especially cellobiohydrolase II: CBIHII (Patent Document 6, for example)) derived from Trichoderma reesei can be used, but there is no limitation thereto.

β-Glucosidase is an exo-type hydrolytic enzyme that liberates glucose units from the non-reducing terminus of cellulose (“glucose unit cleaving”). β-Glucosidase can cleave a β1,4-glycosidic linkage between aglycone or a sugar chain and β-D-glucose, and hydrolyze cellobiose or cello-oligosaccharide, to generate glucose. β-Glucosidase is a typical example of an enzyme that can hydrolyze cellobiose or cello-oligosaccharide. Currently, there is one type of known β-glucosidase and it is referred to as β-glucosidase 1. For example β-glucosidase (especially β-glucosidase 1: BGL1 (Non-Patent Document 7, for example)) derived from Aspergillus aculeatus can be used, but there is no limitation thereto.

For favorable cellulose hydrolysis, enzymes that hydrolyze cellulose in different ways may be used in combination. Various enzymes that hydrolyze cellulose in different ways, such as intramolecular cellulose cleaving, cellulose molecule terminal cleaving, and glucose unit cleaving, may be used in combination as appropriate. Examples of the enzymes that have the respective ways of hydrolysis include endoglucanase, cellobiohydrolase, and β-glucosidase, but there is no limitation thereto. A combination of enzymes that hydrolyze cellulose in different ways may be selected from the group consisting of endoglucanase, cellobiohydrolase, and β-glucosidase, for example. Since it is desirable that glucose, which is a constituent sugar of cellulose, is eventually produced, at least one enzyme that can generate glucose is preferably included. Regarding the enzyme that can generate glucose, endoglucanase as well as glucose unit cleaving enzyme (e.g., β-glucosidase) can generate glucose. For example, β-glucosidase, endoglucanase, and cellobiohydrolase can be secreted by, or displayed on the surface of a yeast.

Terminator DNA

The polynucleotide containing a secretion cassette or a surface display cassette can further contain a terminator DNA.

It is sufficient that the terminator DNA has terminator activity. The “terminator activity” refers to activity that terminates transcription in a terminator region. The terminator needs only to have terminator activity, and can be cut out from cells, phages, and the like harboring a desired terminator region, using a restriction enzyme. A DNA fragment of a terminator region can be obtained by amplifying a desired terminator region by PCR using a primer provided with a restriction enzyme recognition site or a site for insertion into a cloning vector as necessary. Furthermore, a desired terminator DNA may be chemically synthesized based on base sequence information of an already known terminator region.

Examples of the terminator DNA include an a-agglutinin terminator, an ADH1 (aldehyde dehydrogenase) terminator, a TDH3 (glyceraldehyde-3′-phosphate dehydrogenase) terminator, and a DIT1 terminator.

Construction of Expression Cassette

An “expression cassette” as used herein refers to a DNA sequence or a polynucleotide in which a DNA encoding an intended protein and various regulatory elements for regulating the expression of the intended protein are linked to each other in a state of being capable of functioning in host microorganisms or host cells so that the intended protein can be expressed. Here, the term “linked to each other in a state of being capable of functioning” means that the constituents contained in an expression cassette or an expression vector are linked to each other so that a DNA encoding an intended protein is expressed under the control of a promoter and optionally under the control of other regulatory elements. The constituents may also be linked to each other while further containing a sequence of a linker or the like therebetween as long as the intended protein can be expressed.

Examples of the expression cassette include a secretion-type expression cassette and a surface display-type expression cassette as described below.

The secretion-type expression cassette contains the promoter DNA, a DNA encoding the secretion signal peptide of the present invention, and a DNA encoding the intended protein (this expression cassette is also referred to as “secretion cassette”).

The surface display-type expression cassette contains the promoter DNA, a DNA encoding the secretion signal peptide of the present invention, a DNA encoding the intended protein, and a DNA encoding the anchor domain (this expression cassette is also referred to as “surface display cassette”).

In the secretion cassette, the promoter DNA, the DNA encoding the secretion signal peptide, and the DNA encoding the intended protein can be linked to each other in a state of being capable of functioning in host microorganisms or host cells. The secretion cassette is constructed so as to contain the promoter DNA, the DNA encoding the secretion signal peptide of the present invention, and the DNA encoding the intended protein in the stated order in a 5′ to 3′ direction, for example. The secretion cassette can further contain the terminator DNA downstream of the DNA encoding the intended protein.

In the surface display cassette, the promoter DNA, the DNA encoding the secretion signal peptide, the DNA encoding the intended protein, and the DNA encoding the anchor domain can be linked to each other in a state of being capable of functioning in host microorganisms or host cells. The surface display cassette is constructed so as to contain the promoter DNA, the DNA encoding the secretion signal peptide of the present invention, the DNA encoding the intended protein, and the DNA encoding the anchor domain in the stated order in a 5′ to 3′ direction, for example. The surface display cassette can further contain the terminator DNA downstream of the DNA encoding the anchor domain.

DNAs having various sequences can be synthesized and bound using a technique that can be usually used by a person skilled in the art. For example, the binding of the constituents can be performed by cleaving the constituent DNAs provided with an appropriate restriction enzyme recognition sequence by PCR using the restriction enzyme, followed by ligating them using a ligase or the like, or by using one-step isothermal assembly (Non-Patent Document 8). The use of these methods enables a secretion signal peptide to be accurately cleaved and an active enzyme to be expressed.

Also, the expression cassette can be used by excising the region (structural gene) encoding the intended protein (e.g., enzymes such as endoglucanase, cellobiohydrolase, and β-glucosidase, fluorescent proteins, or various antibodies) and the expression regulatory sequences such as the promoter and the terminator as appropriate in a form suitable for vector preparation from each plasmid containing each of them, and then preparing an insert.

Alternatively, the expression cassette (the secretion cassette or the surface display cassette) of the present invention may contain a cloning site for insertion of the DNA encoding the intended protein instead of containing the DNA encoding the intended protein. Such a cloning site is known in the art, and a multicloning site containing sites recognized by various restriction enzymes can be used, for example. Using the expression cassette containing such a cloning site facilitates the insertion of the DNA encoding the intended protein into the expression cassette via the cloning site.

Expression Vector

The present invention provides an expression vector containing the above-mentioned expression cassette. The secretion-type expression vector of the present invention contains the above-mentioned secretion-type expression cassette, and the surface display-type expression vector of the present invention contains the above-mentioned surface display-type expression cassette. The “expression vector” as used herein refers to a vector into which a unit (expression cassette) for the expression of the DNA encoding the intended protein is inserted, and includes a vector into which the DNA encoding the intended protein is inserted. A plasmid vector or an artificial chromosome may be used as the expression vector. If a yeast is used as a host, the vector is preferably in the form of a plasmid because a vector can be easily prepared and a yeast cell can be easily transformed. In order to simplify the procedure for obtaining a DNA, the vector is preferably a shuttle vector between a yeast and Escherichia coli. The vector can contain regulatory sequences (e.g., operator and enhancer) as necessary. Such a vector has a replication origin (Ori) of a 2 μm plasmid of a yeast and a replication origin of ColE1, for example, as well as a yeast selectable marker (described below) and an Escherichia coli selectable marker (e.g., drug-resistant gene).

Any known markers can be used as the yeast selectable marker. Examples thereof include drug-resistant genes, and auxotrophic marker genes (e.g., a gene encoding imidazoleglycerol-phosphate dehydrogenase (HIS3), a gene encoding beta-isopropyl-malate dehydrogenase (LEU2), a gene encoding O-acetylhomoserine and O-acetylserine sulfhydrylase (MET15), a gene encoding tryptophan synthase (TRP5), a gene encoding argininosuccinate lyase (ARG4), a gene encoding N-(5′-phosphoribosyl) anthranilate isomerase (TRP1), a gene encoding histidinol dehydrogenase (HIS4), a gene encoding orotidine-5-phosphate decarboxylase (URA3), a gene encoding dihydroorotate dehydrogenase (URA1), a gene encoding galactokinase (GAL1), and a gene encoding alpha-aminoadipate reductase (LYS2)). For example, the auxotrophic marker genes (e.g., HIS3, LEU2, URA3, and MET15 deficient markers) can be preferably used.

Preparation of Transformed Yeast

There is no particular limitation on the yeast to be used as a host as long as it belongs to Ascomycetous yeasts. Examples thereof include yeasts belonging to the genus Saccharomyces, the genus Kluyveromyces, the genus Candida, the genus Pichia, the genus Schizosaccharomyces, the genus Hancenula, the genus Kloeckera, the genus Schwanniomyces, the genus Komagataella, and the genus Yarrowia.

The yeast of the present invention is obtained by introducing the above-mentioned expression cassette or expression vector into a host yeast. To “introduce” includes not only to introduce a gene (DNA encoding the intended protein) intended to be expressed in the expression cassette or the expression vector into a host cell but also to allow it to be expressed in the host cell. There is no particular limitation on the introducing method, and known methods can be used. A typical example thereof is a method for transforming a yeast using the above-described expression vector of the present invention. There is no particular limitation on the transformation method, and known methods including transfection methods such as a calcium phosphate method, an electroporation method, a lipofection method, a DEAE dextran method, a lithium acetate method, and a protoplast method, and microinjection methods can be used without limitation. The introduced gene may be present in the form of a plasmid, or may be present in the form inserted into a yeast chromosome, or in the form incorporated into a yeast chromosome by homologous recombination.

A yeast secreting a protein can be produced by introducing the expression vector containing the secretion cassette into a yeast to obtain the transformed yeast. A yeast displaying a protein on its surface can be produced by introducing the expression vector containing the surface display cassette into a yeast to obtain the transformed yeast.

The yeast into which the above-mentioned expression cassette or expression vector has been introduced can be selected according to a commonly used method, using, as an indicator, the character with the yeast selectable marker, the activity of the intended protein in the cells (in the case of the surface display-type), the activity of the intended protein outside the cells (in the case of the secretion-type), or the like.

Furthermore, a regular method can be used to confirm that the intended protein is immobilized on the cell surface (displayed on the cell surface) of the transformed yeast obtained by introducing the expression vector containing the surface display cassette.

For example, a subject yeast is allowed to act on an antibody against this protein and a secondary antibody labeled with a fluorescent molecule such as FITC or a secondary antibody labeled with an enzyme such as alkaline phosphatase, or is allowed to act on an antibody against this protein and a biotin-labeled secondary antibody, followed by a fluorescence-labeled streptavidin.

A yeast can also be transformed so that a plurality of proteins are expressed so as to be secreted or displayed on the cell surface. In this case, expression vectors containing respective gene expression cassettes having respective sequences encoding a plurality of types of proteins may be constructed, or a plurality of gene expression cassettes can also be contained in one expression vector. For example, a vector for simultaneous expression of three genes, pATP403, can be used (Non-Patent Document 9).

The transformed yeast of the present invention can be cultured in culture conditions that can be generally used for yeasts. The transformed yeast can be cultured as appropriate using a method well known to a person skilled in the art. The composition of a culture medium, the culture pH, and the culture temperature can be determined as appropriate according to the characteristics of the yeast and the intended protein. The cell density during the culture, and the culture time can also be determined as appropriate according to the characteristics of the yeast and the intended protein.

Furthermore, according to the present invention, a method of protein secretory production is also provided. This method can be carried out using the secretion-type transformed yeast in the above-described culture step. For example, the secretory production of a protein such as an antibody or an enzyme can be performed using this method. A step of collecting a product containing fraction from the culture solution, and in addition, a step of purifying or concentrating this fraction can also be carried out as necessary. These steps and means required in the steps are selected as appropriate by a person skilled in the art.

When the transformed yeast of the present invention is subjected to fermentation culture, culture conditions that are generally used to yeasts can be selected as appropriate and used. Typically, in culture for fermentation, stationary culture, shaking culture, aerated and stirred culture, or the like can be used. The aeration condition can be selected as appropriate from anaerobic conditions, microaerobic conditions, aerobic conditions, and the like. The composition of a culture medium, the culture pH, the culture temperature, the cell density during the culture, and the culture time, and subsequent collection, purification and concentration are determined as appropriate.

### EXAMPLES

Hereinafter, the present invention will be described by way of examples, but the present invention is not limited thereto.

The amino acid sequences of an SED1 secretion signal (SEQ ID No. 2) and a CWP2 secretion signal (SEQ ID No. 20) were extracted based on the prediction made by a signal peptide prediction program PSORT (http://psort.nibb.ac.jp/) (which can be used as WoLF PSORT (http://www.genscript.com/psort/wolf₁₃psort.html)) using the amino acid sequences encoded by the respective genes accepted in GenBank.

The yeast Saccharomyces cerevisiae BY4741 strain (Non-Patent Document 8) used in the examples was obtained from Invitrogen.

All PCR methods shown in the examples were performed using KOD-Plus-Neo-DNA polymerase (manufactured by Toyobo Co., Ltd.).

All gene introductions to yeasts shown in the examples were performed according to the lithium acetate method.

### Preparation Example 1

### Preparation of Vector Plasmids Containing Various Expression Cassettes

Plasmids containing respective expression cassettes X1 to X19 below were prepared.

Xl: SED1 promoter+glucoamylase secretion signal+BGL1+SED1 anchor domain

X2: SED1 promoter+SED1 secretion signal+BGL1+SED1 anchor domain

X3: SED1 promoter+MFα prepro+BGL1+SED1 anchor domain

X4: SED1 promoter+HKR1 secretion signal+BGL1+SED1 anchor domain

X5: SED1 promoter+glucoamylase secretion signal+BGL1

X6: SED1 promoter+SED1 secretion signal+BGL1

X7: SED1 promoter+MFα prepro+BGL1

X8: SED1 promoter+HKR1 secretion signal+BGL1

X9: SED1 promoter+glucoamylase secretion signal+EGII+SED1 anchor domain

X10: SED1 promoter+SED1 secretion signal+EGII+SED1 anchor domain

X11: SED1 promoter+MFα prepro+EGII+SED1 anchor domain

X12: TDH3 promoter+glucoamylase secretion signal+BGL1+SED1 anchor domain

X13: TDH3 promoter+SED1 secretion signal+BGL1+SED1 anchor domain

X14: TDH3 promoter+MFα prepro+BGL1+SED1 anchor domain

X15: PGK1 promoter+glucoamylase secretion signal+BGL1+SED1 anchor domain

X16: PGK1 promoter+SED1 secretion signal+BGL1+SED1 anchor domain

X17: PGK1 promoter+MFα prepro+BGL1+SED1anchor domain

X18: CWP2 promoter+SED1 secretion signal+BGL1

X19: CWP2 promoter+CWP2 secretion signal+BGL1

X20: TDI-11 promoter derived from Pichia pastoris+MFα prepro+GFP

X21: TDI-11 promoter derived from Pichia pastoris+glucoamylase secretion signal+GFP

X22: TDH1 promoter derived from Pichia pastoris+SED1 secretion signal+GFP

The base sequences and amino acid sequences of the constituents of the above-mentioned expression cassettes are as follows.

SEQ ID No. 1: base sequence of a DNA encoding the secretion signal peptide of SED1 derived from Saccharomyces cerevisiae

SEQ ID No. 2: amino acid sequence of the secretion signal peptide of SED1 derived from Saccharomyces cerevisiae

SEQ ID No. 3: base sequence of the promoter of SED1 derived from Saccharomyces cerevisiae

SEQ ID No. 4: base sequence of a region in which the initiation codon is excluded from the region of a DNA encoding the SED1 anchor protein derived from Saccharomyces cerevisiae, which was used as the SED1 anchor domain in the examples of the present invention

SEQ ID No. 5: amino acid sequence of the SED1 anchor protein (in which the initiation methionine is not included) derived from Saccharomyces cerevisiae, which was used as the SED1 anchor domain in the examples of the present invention

SEQ ID No. 6: base sequence of a DNA encoding the glucoamylase secretion signal peptide derived from Rhizopus oryzae

SEQ ID No. 7: amino acid sequence of the glucoamylase secretion signal peptide derived from Rhizopus ozyzae

SEQ ID No. 8: base sequence of a DNA encoding the MFα prepro derived from Saccharomyces cerevisiae

SEQ ID No. 9: amino acid sequence of the MFα prepro derived from Saccharomyces cerevisiae

SEQ ID No. 10: base sequence of a DNA encoding the secretion signal peptide of a secretion protein HKR1 derived from Saccharomyces cerevisiae

SEQ ID No. 11: amino acid sequence of the secretion signal peptide of a secretion protein HKR1 derived from Saccharomyces cerevisiae

SEQ ID No. 12: base sequence of the TDH3 promoter derived from Saccharomyces cerevisiae

SEQ ID No. 13: base sequence of the PGK1 promoter derived from Saccharomyces cerevisiae

SEQ ID No. 14: base sequence of a DNA encoding β-glucosidase 1 (BGL1) derived from Aspergillus aculeatus

SEQ ID No. 15: amino acid sequence of β-glucosidase 1 (BGL1) derived from Aspergillus aculeatus

SEQ ID No. 16: base sequence of a DNA encoding endoglucanase II (EGII) derived from Trichoderma reesei

SEQ ID No. 17: amino acid sequence of endoglucanase II (EGII) derived from Trichoderma reesei

SEQ ID No. 18: base sequence of the CWP2 promoter derived from Saccharomyces cerevisiae

SEQ ID No. 19: base sequence of a DNA encoding the CWP2 secretion signal peptide derived from Saccharomyces cerevisiae

SEQ ID No. 20: amino acid sequence of the CWP2 secretion signal peptide derived from Saccharomyces cerevisiae

SEQ ID No. 21: base sequence of the TDH1 promoter derived from Pichia pastoris

SEQ ID No. 22: base sequence of a DNA encoding Umikinoko-Green 1 (mUkG1) optimized for the codon usage in Pichia pastoris

SEQ ID No. 23: amino acid sequence of Umikinoko-Green 1 (mUkG1)

Hereinafter, procedures for preparing various plasmids used to obtain the transformed yeast strains that were used in the examples will be described.

A DNA fragment containing the promoter region and the secretion signal sequence of a cell surface-localized protein gene Sed1 derived from Saccharomyces cerevisiae was prepared through amplification by PCR from a Saccharomyces cerevisiae BY4741 strain genome as a template using a primer pair of Sed1p-F (SEQ ID No. 24) and Sed1ss-R (SEQ ID No. 25). By use of One-step isothermal assembly, this fragment was ligated to a fragment amplified using a vector plasmid pIBG-SGS (a surface expression vector (corresponding to pIBG-SS in Non-Patent Document 10) including an auxotrophic marker gene HES3 and a BGL1 expression cassette (i.e., cassette in which the SED1 promoter, the secretion signal peptide sequence of glucoamylase derived from Rhizopus oryzae, the coding region of the β-glucosidase 1 (BGL1) gene derived from Aspergillus aculeatus, the coding region of the SED1 gene, and the terminator region 445 bp downstream of the coding region of the a-agglutinin gene were arranged in this order: expression cassette X1)) as a template, and using a primer pair of BGL1-F (SEQ ID No. 26) and PRS-R (SEQ ID No. 27). The plasmid containing expression cassette X2 thus obtained was named pIBG-SSS.

A fragment containing the MFα prepro leader sequence derived from Saccharomyces cerevisiae was prepared through amplification by PCR from pIUPGSBAAG (a vector for surface expression of α-amylase including the MFα prepro sequence and the 3′ -half of the α-agglutinin gene (Non-Patent Document 11)) as a template using a primer pair of MFα-F (SEQ ID No. 28) and MFα-R (SEQ ID No. 29). By use of One-step isothermal assembly, this fragment was ligated to a fragment amplified from the vector plasmid pIBG-SGS as a template using a primer pair of BGL1-F2 (SEQ ID No. 30) and Sed1p-R (SEQ ID No. 31). The plasmid containing expression cassette X3 thus obtained was named pIBG-SMS.

A DNA fragment containing the secretion signal sequence of a secretion protein gene Hkr1 derived from Saccharomyces cerevisiae was prepared through amplification by PCR from the Saccharomyces cerevisiae BY4741 strain genome as a template using a primer pair of Hkr1ss-F (SEQ ID No. 32) and (SEQ ID No. 33). By use of One-step isothermal assembly, this fragment was ligated to a fragment amplified from the vector plasmid pIBG-SGS as a template using a primer pair of BGL1-F3 (SEQ ID No. 34) and Sed1p-R2 (SEQ ID No. 35). The plasmid containing expression cassette X4 thus obtained was named pIBG-SHS.

Fragments that have respective sequences of pIBG-SGS, pIBG-SSS, pIBG-SMS, and pIBG-SHS from which the coding region of the SED1 gene was removed were prepared through amplification from the respective plasmids as templates using a primer pair of PRS-BsrGI-F (SEQ ID No. 36) and BGL1-BsrGI-R (SEQ ID No. 37). These fragments were treated with BsrGI, and then circularized using a self ligation method. The plasmids containing expression cassettes X5, X6, X7, and X8 thus obtained were named pIBG-SGsec, pIBG-SSsec, pIBG-SMsec, and pIBG-SHsec, respectively.

The coding region of the endoglucanase II (EGII) gene derived from Trichoderma reesei was prepared through amplification by PCR from pIEG-SGS (a surface expression vector (corresponding to pIEG-SS in Non-Patent Document 10) including the auxotrophic marker gene HIS3 and an EGII expression cassette (i.e., cassette in which the SED1 promoter, the secretion signal peptide sequence of glucoamylase derived from Rhizopus oryzae, the coding region of the EGII gene, the coding region of the SED1 gene, and the terminator region 445 bp downstream of the coding region of the α-agglutinin gene were arranged in this order: expression cassette X9)) as a template using a primer pair of EGII-F (SEQ ID No. 38) and EGII-R (SEQ ID No. 39). By use of One-step isothermal assembly, this fragment was ligated to a fragment amplified from the vector plasmid pIBG-SSS as a template using a primer pair of Sed1a-F (SEQ ID No. 40) and Sed1ss-R2 (SEQ ID No. 41). The plasmid containing expression cassette X10 thus obtained was named pIEG-SSS.

The coding region of the EGII gene was prepared through amplification by PCR from pIEG-SGS as a template using a primer pair of EGII-F2 (SEQ ID No. 42) and EGII-R (SEQ ID No. 39). By use of One-step isothermal assembly, this fragment was ligated to a fragment amplified from the vector plasmid pIBG-SMS as a template using a primer pair of Sed1a-F (SEQ ID No. 40) and MFα-R2 (SEQ ID No. 43). The plasmid containing expression cassette X11 thus obtained was named pIEG-SMS.

The promoter region of the gene TDH3 derived from Saccharomyces cerevisiae was prepared through amplification by PCR from pIBG-TGS (a surface expression vector (corresponding to pIBG-TS in Non-Patent Document 10) including the auxotrophic marker gene HIS3 and an BGL1 expression cassette (i.e., cassette in which the TDH3 promoter, the secretion signal peptide sequence of glucoamylase derived from Rhizopus oryzae, the coding region of the BGL1 gene, the coding region of the SED1 gene, and the terminator region 445 bp downstream of the coding region of the a-agglutinin gene were arranged in this order: expression cassette X12)) as a template using a primer pair of TDH3p-F (SEQ ID No. 44) and TDH3p-R (SEQ ID No. 45). By use of One-step isothermal assembly, this fragment was ligated to a fragment amplified from the vector plasmid pIBG-SSS as a template using a primer pair of Sed1ss-F (SEQ ID No. 46) and PRS-R2 (SEQ ID No. 47). The plasmid containing expression cassette X13thus obtained was named pIBG-TSS.

The promoter region of the gene TDH3 derived from Saccharomyces cerevisiae was prepared through amplification by PCR from pIBG-TGS as a template using a primer pair of TDH3p-F (SEQ ID No. 44) and TDH3p-R2 (SEQ ID No. 48). By use of One-step isothermal assembly, this fragment was ligated to a fragment amplified from the vector plasmid pIBG-SMS as a template using a primer pair of MFα-F2 (SEQ ID No. 49) and PRS-R2 (SEQ ID No. 47). The plasmid containing expression cassette X14 thus obtained was named pIBG-TMS.

The promoter region of the gene PGK1 derived from Saccharomyces cerevisiae was prepared through amplification by PCR from pGK403 (a protein expression vector containing a promoter region and a terminator region of PGK1 (Non-Patent Document 12)) as a template using a primer pair of PGK1p-F (SEQ ID No. 50) and PGK1p-R (SEQ ID No. 51). By use of One-step isothermal assembly, this fragment was ligated to a fragment amplified from the vector plasmid pIBG-SGS as a template using a primer pair of GAss-F (SEQ ID No. 52) and PRS-R3 (SEQ ID No. 53). The plasmid containing expression cassette X15 thus obtained was named pIBG-PGS.

The promoter region of the gene PGK1 derived from Saccharomyces cerevisiae was prepared through amplification by PCR from pGK403 as a template using a primer pair of PGK1p-F (SEQ ID No. 50) and PGK1p-R2 (SEQ ID No. 54). By use of One-step isothermal assembly, this fragment was ligated to a fragment amplified from the vector plasmid pIBG-SSS as a template using a primer pair of Sed1ss-F2 (SEQ ID No. 55) and PRS-R3 (SEQ ID No. 53). The plasmid containing expression cassette X16 thus obtained was named pIBG-PSS.

The promoter region of the gene PGK1 derived from Saccharomyces cerevisiae was prepared through amplification by PCR from pGK403 as a template using a primer pair of PGK1p-F (SEQ ID No. 50) and PGK1p-R3 (SEQ ID No. 56). By use of One-step isothermal assembly, this fragment was ligated to a fragment amplified from the vector plasmid pIBG-SMS as a template using a primer pair of MFα-F3 (SEQ ID No. 57) and PRS-R3 (SEQ ID No. 53). The plasmid containing expression cassette X17 thus obtained was named pIBG-PMS.

The promoter region of a cell surface-localized protein gene CWP2 derived from Saccharomyces cerevisiae was prepared through amplification by PCR from the Saccharomyces cerevisiae BY4741 strain genome as a template using a primer pair of Cwp2p-F (SEQ ID No. 58) and Cwp2p-R (SEQ ID No. 59). By use of One-step isothermal assembly, this fragment was ligated to a fragment amplified from the vector plasmid pIBG-SSsec as a template using a primer pair of Sed1ss-F3 (SEQ ID No. 60) and PRS-R4 (SEQ ID No. 61). The plasmid containing expression cassette X18 thus obtained was named pIBG-CSsec.

A fragment containing the promoter region and the secretion signal. sequence of the cell surface-localized protein gene CWP2 derived from Saccharomyces cerevisiae was prepared through amplification by PCR from the Saccharomyces cerevisiae BY4741 strain genome as a template using a primer pair of Cwp2p-F (SEQ ID No. 58) and Cwp2ss-R (SEQ ID No. 62). By use of One-step isothermal assembly, this fragment was ligated to a fragment amplified from the vector plasmid pIBG-SSsec as a template using a primer pair of BGL1-F4 (SEQ ID No. 63) and PRS-R4 (SEQ ID No. 61). The plasmid containing expression cassette X19 thus obtained was named pIBG-CCsec.

Gene synthesis was performed to obtain a fragment in which the promoter region of the TDH1 gene derived from Pichia pastoris, an SpeI site, the MFα prepro leader sequence derived from Saccharomyces cerevisiae, an XhoI site, the coding region of a green fluorescent protein (GFP), and a terminator region of an AOX1 gene derived from Pichia pastoris were arranged in this order and a Hindill site and a BamHI site were provided at both termini. As the GFP gene, an Umikinoko-Green 1 (mUkG1) gene optimized for the codon usage of Pichia pastoris was used. This fragment was treated with HindIII and BamHI and ligated to a plasmid pUC19 treated in the same manner. The obtained plasmid was named pmUkG1_MFα. Subsequently, a G418 resistance gene sequence was prepared through amplification by PCR from a plasmid pPIC9K manufactured by Life technology as a template using a primer pair of G418r-BamHI-F (SEQ ID No. 64) and G418r-EcoRI-R (SEQ ID No. 65). This fragment was treated with BamHI and EcoRI and ligated to the plasmid pmUkG1_MFα treated in the same manner. The plasmid containing expression cassette X20 thus obtained was named pGmUkG1_MFα.

The secretion signal peptide sequence of glucoamylase derived from Rhizopus oryzae was prepared through amplification by PCR from pIBG-SGS as a template using a primer pair of MFα-SpeI-F (SEQ ID No. 66) and MFα-XhoI-R (SEQ ID No. 67). This fragment was treated with SpeI and XhoI and ligated to the plasmid pGmUkG1_MFα treated in the same manner from which the MFα prepro leader sequence was removed. The plasmid containing expression cassette X21 thus obtained was named pGmUkG1_GA.

The secretion signal peptide sequence of SED1 derived from Saccharomyces cerevisiae was prepared through amplification by PCR from the Saccharomyces cerevisiae BY4741 strain genome as a template using a primer pair of Sed1ss-SpeI-F (SEQ ID No. 68) and Sed1ss-XhoI-R (SEQ ID No. 69). This fragment was treated with SpeI and XhoI and ligated to the plasmid pGmUkG1_MFα treated in the same manner from which the MFα prepro leader sequence was removed. The plasmid containing expression cassette X22 thus obtained was named pGmUkG1_SED1.

### Preparation Example 2

### Preparation of Various Transformed Yeasts

The plasmids (pIBG-SGS, pIBG-SSS, pIBG-SMS, pIBG-SHS, pIBG-SGsec, pIBG-SSsec, pIBG-SMsec, pIBG-SHsec, pIEG-SGS, pIEG-SSS, pIEG-SMS, pIBG-TGS, pIBG-TSS, pIBG-TMS, pIBG-PGS, pIBG-PSS, pIBG-PMS, pIBG-CSsec, and pIBG-CCsec) described in Preparation Example 1 were treated with NdeI and used to transform the yeast Saccharomyces cerevisiae BY4741 strain (MATα his3 leu2 met15 ura3 strain) using the lithium acetate method. These transformants are referred to as a BY-BG-SGS strain, a BY-BG-SSS strain, a BY-BG-SMS strain, a BY-BG-SHS strain, a BY-BG-SGsec strain, a BY-BG-SSsec strain, a BY-BG-SMsec strain, a BY-BG-SHsec strain, a BY-EG-SGS strain, a BY-EG-SSS strain, a BY-EG-SMS strain, a BY-BG-TGS strain, a BY-BG-TSS strain, a BY-BG-TMS strain, a BY-BG-PGS strain, a BY-BG-PSS strain, a BY-BG-PMS strain, a BY-BG-CSsec strain, and a BY-BG-CCsec strain, respectively.

The plasmids (pGmUkG1_MFα, pGmUkG1_GA, and pGmUkG1_SED1) described in Preparation Example 1 were treated with BsiWI and used to transform yeast Pichia pastoris CBS7435 strain (wild-type strain) using the lithium acetate method. These transformants are referred to as a PP-GFP-MFα strain, a PP-GFP-GA strain, and a PP-GFP-SED1 strain, respectively.

### Test Example 1

### Examination of 6-Glucosidase Activity (BGL)

In the case of the surface display strains, the BGL activity of the cells (activity (U) per weight of dry cells) was measured, and in the case of the secretion strains, the BGL activity in the culture media (activity (U) per litter of culture medium) was measured. The β-glucosidase (BGL) activity was examined according to the following procedure.

Cells were transplanted to 5 mL of SD medium (supplemented with leucine, methionine, and uracil) and cultured at 30° C. and 180 rpm for 18 hours (pre-culture), and then were transplanted to 50 mL of 1×YPD medium (initial OD₆₀₀=0.05) and cultured at 30° C. and 150 rpm (main culture). The culture solutions were collected every 24 hours after the start of the main culture, and then centrifuged at 1,000 g for 5 minutes so as to separate the cells and the culture medium.

The β-glucosidase activity of the cells was measured as follows:

(1) the cells were washed twice with distilled water;

(2) 500 μL of reaction liquid (composition: 100 μL of 10 mM pNPG (p-nitrophenyl-β-D-glucopyranoside) (final concentration 2 mM); 50 μL of 500 mM sodium citrate buffer solution (pH 5.0) (final concentration 50 mM); 250 μL of distilled water; and 100 μL of yeast suspension (final cell concentration of 1 to 10 g wet cells/L)) was prepared and allowed to react at 500 rpm and 30° C. for 10 minutes;

(3) after the end of the reaction, the reaction was stopped by adding 500 μL of 3 M Na₂CO₃; and

(4) the liquid was centrifuged at 10,000 g for 5 minutes, and then the absorbance ABS₄₀₀ at 400 nm of the supernatant was measured. The amount of enzyme that liberates 1 μmol of pNP (p-nitrophenol) in 1 minute is taken as 1 U.

The BGL activity in the culture medium was measured in the same manner as describe above, except that 100 μL of the culture medium was used instead of the yeast suspension in the preparation of the above-mentioned reaction liquid.

### Test Example 2

### Examination of Endoglucanase (EG) Activity

The EG activity of the cells of the surface display strains (activity (U) per weight of dry cells) was examined according to the following procedure.

Cells were transplanted to 5 mL of SD medium (supplemented with leucine, methionine, and uracil) and cultured at 30° C. and 180 rpm for 18 hours (pre-culture), and then were transplanted to 50 mL of 1×YPD medium (initial OD₆₀₀=0.05) and cultured at 30° C. and 150 rpm (main culture). The culture solutions were collected 48 hours after the start of the main culture, and then centrifuged at 1,000 g for 5 minutes so as to separate the cells and the culture medium.

The endoglucanase activity of the cells was measured as follows:

(1) the cells were washed twice with distilled water;

(2) 2500 μL of reaction liquid (composition: one tablet of CELLAZYME C (manufactured by Megazyme); 250 μL of 500 mM sodium citrate buffer solution (pH 5.0) (final concentration 50 mM); 2000 μL of distilled water; and 250 μL of yeast suspension (final cell concentration of 10 g wet cells/L)) was prepared, and allowed to stand to react at 38° C. for 4 hours; and

(3) after the end of the reaction, the liquid was centrifuged at 10,000 g for 5 minutes, and then the supernatant was measured by the absorbance at 590 nm, ABS₅₉₀.

### Test Example 3

### Examination of Secretory Expression of GFP in Pichia pastoris

For GFP secretion strains of Pichia pastoris, the GFP fluorescence intensity in the culture media was according to the following procedure.

Cells were transplanted to 500 μL of BMGY medium in a 96-deep-well plate and cultured at 30° C. and 1800 rpm for 18 hours (pre-culture), and then pre-culture solutions each were transplanted to 500 μL of fresh BMGY medium in a 96-deep-well plate and cultured at 30° C. and 1800 rpm (main culture). The 96-deep-well plate was centrifuged at 3,000 rpm for 5 minutes 24 hours after the start of the main culture, and the culture supernatant in each well was obtained.

To a 96-well-black plate, 100 μL of the culture supernatant was added, and the GFP fluorescence intensity (excitation wavelength: 485 nm: fluorescent wavelength: 510 nm) in each well was measured using EnVison 2104 Multilabel Reader (manufactured by Perkin Elmer).

### Example 1

### Comparison of Various Secretion Signals in Surface Display of β-Glucosidase

In this example, the BGL activity of the cells was measured for various transformed surface display yeasts (a BY-BG-SGS strain, a BY-BG-SSS strain, a BY-BG-SMS strain, and a BY-BG-SHS strain) which were obtained by introducing the plasmids containing expression cassettes X1 to X4, respectively.

FIG. 1 shows the results. In FIG. 1, the horizontal axis indicates the culture time (“Time (hour)”), and the vertical axis indicates the β-glucosidase activity (activity per weight of dry cells (“U/g dry cell weight”)). Symbols in FIG. 1 are as follows: black circles, SED1 secretion signal (SED1); black rhombuses, glucoamylase secretion signal (GA) derived from Rhizopus oryzae; black triangles, MFα prepro (MFα); and black squares, HKR1 secretion signal (HKR1).

It was found that, as shown in FIG. 1, the transformed BGL surface display strain using the SED1 secretion signal exhibited considerably high surface BGL activity compared with the transformed strains using the MFα prepro, which is often used in the conventional highly efficient expression of a secretory protein, and the glucoamylase secretion signal derived from Rhizopus oryzae, which is often used to express a protein so as to be displayed on the surface.

### Example 2

### Comparison of Various Secretion Signals in Secretion of β-Glucosidase

In this example, the BGL activity in the culture media was measured for various transformed secretion yeasts (a BY-BG-SGsec strain, a BY-BG-SSsec strain, a BY-BG-SMsec strain, and a BY-BG-SHsec strain) which were obtained by introducing the plasmids containing expression cassettes X5 to X8, respectively.

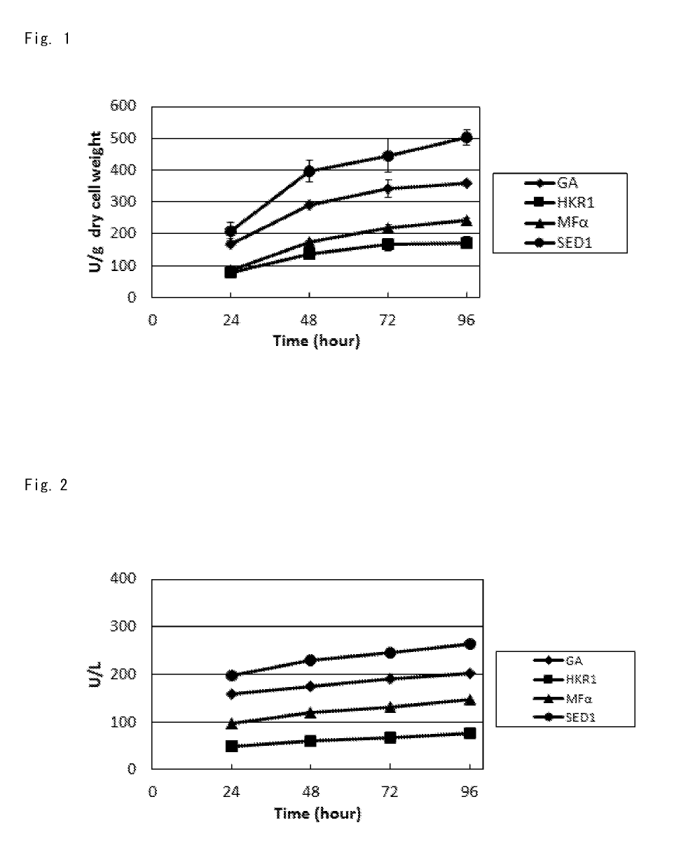

FIG. 2 shows the results. In FIG. 2, the horizontal axis indicates the culture time (“Time (hour)”), and the vertical axis indicates the β-glucosidase activity (activity in culture medium (“U/L”)). Symbols in FIG. 2 are as follows: black circles, SED1 secretion signal (SED1); black rhombuses, glucoamylase secretion signal (GA); black triangles, MFα prepro (MFα); and black squares, HKR1 secretion signal (HKR1).

It was found that, as shown in FIG. 2, the transformed BGL secretion strain using the SED1 secretion signal exhibited considerably high BGL activity compared with the transformed strains using the MFα prepro, which is often used in the conventional highly efficient expression of a secretory protein, and the glucoamylase secretion signal derived from Rhizopus oryzae, which is often used to express a protein so as to be displayed on the surface

### Example 3

### Comparison of Various Secretion Signals in Surface Display of Endoglucanase

In this example, the EG activity of the cells was measured for various transformed surface display yeasts (a BY-EG-SGS strain, a BY-EG-SSS strain, and a BY-EG-SMS strain) which were obtained by introducing the plasmids containing expression cassettes X9 to X11, respectively.

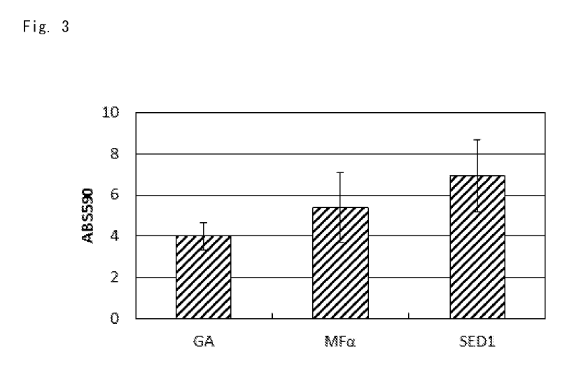

FIG. 3 shows the results. In FIG. 3, the vertical axis indicates the EG activity (absorbance measurement value (“ABS590”)). The bar graphs in FIG. 3 indicate the results of the respective cases of the glucoamylase secretion signal (GA) derived from Rhizopus ozyzae, the MFα prepro (MFα), and the SED1 secretion signal (SED1) in the order from left to right.

As shown in FIG. 3, also in the case where the endoglucanase activity was used as an index, it was observed that the transformed surface display strain using the SED1 secretion signal exhibited higher secretion efficiency than those of the transformed surface display strains using the secretion signals that are conventionally used in many cases.

### Example 4

### Examination of Combination with Various Promoters

In this example, the BGL activity of the cells was measured for various transformed surface display yeasts (a BY-BG-TGS strain, a BY-BG-TSS strain, a BY-BG-TMS strain, a BY-BG-PGS strain, a BY-BG-PSS strain, and a BY-BG-PMS strain) which were obtained by introducing the plasmids containing expression cassettes X12 to X17, respectively.

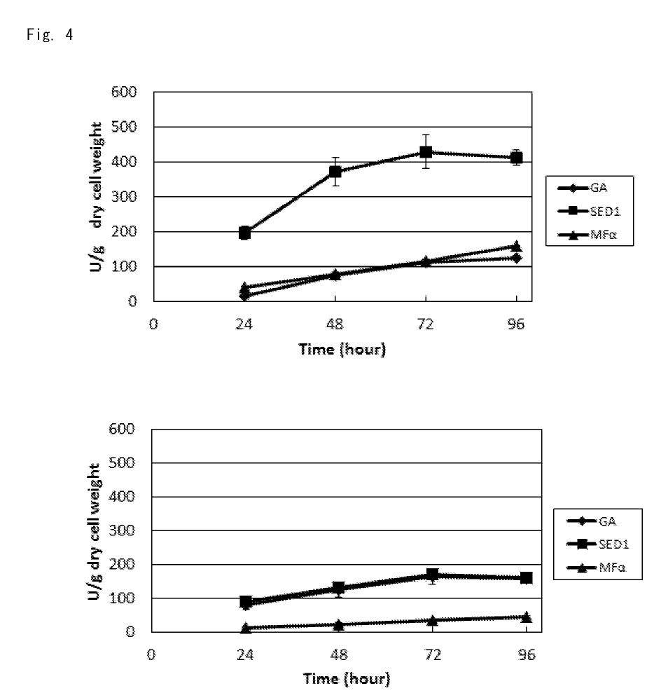

FIG. 4 shows the results (upper graph: TDH3 promoter; and lower graph: PGK1 promoter). In the graphs shown in FIG. 4, the horizontal axis indicates the culture time (“Time (hour)”), and the vertical axis indicates the β-glucosidase activity (activity per weight of dry cells (“U/g dry cell weight”)). Symbols in FIG. 4 are as follows: black squares, SED1 secretion signal (SED1); black rhombuses, glucoamylase secretion signal (GA) derived from Rhizopus oryzae; and black triangles, MFα prepro (MFα).

As shown in FIG. 4, also in the case of the combination with promoters other than the SED1 promoter, it was observed that the strain using the SED1 secretion signal stably exhibited high enzyme activity that was higher than or equal to those of the transformed surface display strains using the secretion signals that are conventionally used in many cases. In particular, in the case of the TDH 3 promoter, a rate of increase in activity is significant in the same manner as in the case of the combination with the SED1 promoter shown in FIG. 1.

### Example 5

### Examination of Combination with Various Promoters

In this example, the BGL activity in the culture media was measured for various transformed secretion yeasts (a BY-BG-CSsec strain and a BY-BG-CCsec strain) which were obtained by introducing the plasmids containing expression cassettes X18 and X19, respectively.

FIG. 5 shows the results. In FIG. 5, the horizontal axis indicates the culture time (“Time (hour)”), and the vertical axis indicates the β-glucosidase activity (activity in culture medium (“U/L”)). Symbols in FIG. 5 are as follows: black rhombuses, CWP2 promoter+SED1 secretion signal; and black squares, CWP2 promoter+CWP2 secretion signal.

As shown in FIG. 5, it was observed that the transformed secretion strain using the CWP2 promoter in combination with the SED1 secretion signal exhibited higher BGL activity than that of the transformed secretion strain using the CWP2 promoter in combination with the CWP2 secretion signal derived from the same gene.

### Example 6

### Examination of Secretion Ability in Different Yeast

In this example, the GFP fluorescence intensity in the culture media was measured for the GFP secretion transformants (referred to as “PP-GFP-MFα strain”, “PP-GFP-GA strain”, and “PP-GFP-SED1 strain”) of yeast Pichia pastoris obtained by introducing the plasmids containing respective expression cassettes X20, X21, and X22, respectively.

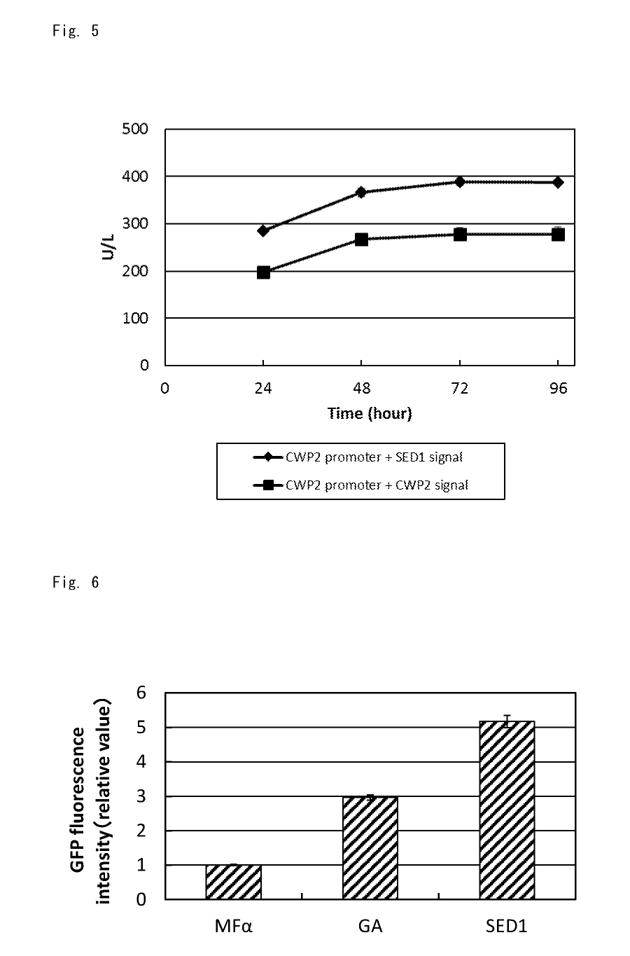

FIG. 6 shows the results. In FIG. 6, the vertical axis indicates the relative value of the GFP fluorescence intensity of the culture media of the strains when the GFP fluorescence intensity of the culture medium of the PP-GFP-MFα strain is taken as 1. The bar graphs in FIG. 6 indicate the results of the respective cases of the MFα prepro (MFα), glucoamylase secretion signal (GA) derived from Rhizopus oryzae, and SED1 secretion signal (SED1) in the order from left to right.

As shown in FIG. 6, also in the case where Pichia pastoris was used as a host, it was observed that the transformed secretion strain using the SED1 secretion signal exhibited considerably higher GFP fluorescence intensity than those of the transformed secretion strains using the secretion signals that are conventionally used in many cases. It was shown from these results that the SED1 secretion signal allowed a protein to be secreted in a larger amount than ever even when a yeast species other than Saccharomyces cerevisiae was used as a host.

### INDUSTRIAL APPLICABILITY

According to the present invention, various types of proteins such as enzymes can be efficiently secreted out of a cell or displayed on the surface of a cell. Accordingly, the present invention is very useful for improvement in efficiency, reduction in cost, and promotion of diffusion of material production by yeasts. It is possible that more specific fields of application are improvement in the efficiency and reduction in cost of secretory production of a protein such as an antibody or an enzyme, production of a chemical product from cellulosic biomass using yeasts that display a cellulose-degrading enzyme on the cell surface, and the like.

1-12. (canceled)

13. An expression vector comprising:
(i) an SED1 promoter DNA;
(ii) a DNA encoding any peptide selected from the group consisting of:
(a) a peptide that has the amino acid sequence of SEQ ID No. 2;
(b) a peptide that has an amino acid sequence having at least 90% sequence identity to the amino acid sequence of SEQ ID No. 2, and has secretion signal activity;
(c) a peptide that has an amino acid sequence obtained by substitution, deletion, or addition of one or several amino acid residues in the amino acid sequence of SEQ ID No. 2 and having at least 90% sequence identity to the amino acid sequence of SEQ ID No. 2, and has secretion signal activity;
(d) a peptide that is encoded by a base sequence having at least 90% sequence identity to the base sequence of SEQ ID No. 1, and has secretion signal activity; and
(e) a peptide that is encoded by a base sequence that can hybridize with a complementary strand of a DNA having the base sequence of SEQ ID No. 1 and has at least 90% sequence identity to the base sequence of SEQ ID No. 1, and has secretion signal activity; and
(iii) a DNA encoding an intended protein or a cloning site for insertion of the DNA encoding an intended protein.

14. The expression vector of claim 13, wherein the item (iii) is a DNA encoding an intended protein.

15. A transformed yeast into which the expression vector of claim 14 has been introduced.

16. A method for producing a yeast performing protein secretory production, the method comprising:
obtaining a transformed yeast by introducing an expression cassette into a yeast, the expression cassette comprising an SED1 promoter DNA; a DNA encoding any peptide selected from the group consisting of (a) a peptide that has the amino acid sequence of SEQ ID No. 2, (b) a peptide that has an amino acid sequence having at least 90% sequence identity to the amino acid sequence of SEQ ID No. 2, and has secretion signal activity, (c) a peptide that has an amino acid sequence obtained by substitution, deletion, or addition of one or several amino acid residues in the amino acid sequence of SEQ ID No. 2 and having at least 90% sequence identity to the amino acid sequence of SEQ ID No. 2, and has secretion signal activity, (d) a peptide that is encoded by a base sequence having at least 90% sequence identity to the base sequence of SEQ ID No. 1, and has secretion signal activity and (e) a peptide that is encoded by a base sequence that can hybridize with a complementary strand of a DNA having the base sequence of SEQ ID No. 1 and has at least 90% sequence identity to the base sequence of SEQ ID No. 1, and has secretion signal activity; and a DNA encoding an intended protein.

17. A method for performing protein secretory production in a yeast, the method comprising:
culturing the transformed yeast of claim 15.

18. An expression vector comprising:
(i) an SED1 promoter DNA;
(ii) a DNA encoding any peptide selected from the group consisting of:
(a) a peptide that has the amino acid sequence of SEQ ID No. 2;
(b) a peptide that has an amino acid sequence having at least 90% sequence identity to the amino acid sequence of SEQ ID No. 2, and has secretion signal activity;
(c) a peptide that has an amino acid sequence obtained by substitution, deletion, or addition of one or several amino acid residues in the amino acid sequence of SEQ ID No. 2 and having at least 90% sequence identity to the amino acid sequence of SEQ ID No. 2, and has secretion signal activity;
(d) a peptide that is encoded by a base sequence having at least 90% sequence identity to the base sequence of SEQ ID No. 1, and has secretion signal activity; and
(e) a peptide that is encoded by a base sequence that can hybridize with a complementary strand of a DNA having the base sequence of SEQ ID No. 1 and has at least 90% sequence identity to the base sequence of SEQ ID No. 1, and has secretion signal activity;
(iii) a DNA encoding an intended protein or a cloning site for insertion of the DNA encoding an intended protein; and
(iv) a DNA encoding an anchor domain.

19. The expression vector of claim 18, wherein the anchor domain is an SED1 anchor domain.

20. The expression vector of claim 18, wherein the item (iii) is a DNA encoding an intended protein.

21. A transformed yeast into which the expression vector of claim 20 has been introduced.

22. A method for producing a yeast displaying a protein on a surface, the method comprising:
obtaining a transformed yeast by introducing an expression cassette into a yeast, the expression cassette comprising an SED1 promoter DNA; a DNA encoding any peptide selected from the group consisting of (a) a peptide that has the amino acid sequence of SEQ ID No. 2, (b) a peptide that has an amino acid sequence having at least 90% sequence identity to the amino acid sequence of SEQ ID No. 2, and has secretion signal activity, (c) a peptide that has an amino acid sequence obtained by substitution, deletion, or addition of one or several amino acid residues in the amino acid sequence of SEQ ID No. 2 and having at least 90% sequence identity to the amino acid sequence of SEQ ID No. 2, and has secretion signal activity, (d) a peptide that is encoded by a base sequence having at least 90% sequence identity to the base sequence of SEQ ID No. 1, and has secretion signal activity and (e) a peptide that is encoded by a base sequence that can hybridize with a complementary strand of a DNA having the base sequence of SEQ ID No. 1 and has at least 90% sequence identity to the base sequence of SEQ ID No. 1, and has secretion signal activity; a DNA encoding an intended protein; and a DNA encoding an anchor domain.

23. A method for performing protein secretory production in a yeast, the method comprising:
culturing a yeast produced using the method of claim 16.

24. The expression vector of claim 19, wherein the item (iii) is a DNA encoding an intended protein.


SEQUENCE LISTING (69 sequences)

SEQ ID NO: 1 - DNA, 57 bp, Saccharomyces cerevisiae
  CDS (1)..(57): SED1 secretory signal
atgaaattatcaactgtcctattatctgccggtttagcctcgactactttggcccaa

SEQ ID NO: 2 - protein, 19 aa, Saccharomyces cerevisiae
MKLSTVLLSAGLASTTLAQ

SEQ ID NO: 3 - DNA, 800 bp, Saccharomyces cerevisiae
  promoter (1)..(800): SED1 promoter
attggatatagaaaattaacgtaaggcagtatcttttcacaatgtacttgcaacgcggcgacttaaagttgaagtacaacctgcagcagcggctttttgtacggtacgccaaactgtcaatggataatattgcgtagaccgaaaaaggtaatcctcaacactacccgtggtggatgacctaaagcagtaatattggttggaattatctcccagacggcaccgtctccccgagaaagcttagccccgaggtctaccttccatacaccactgattgctccacgtcatgcggccttctttcgaggacaaaaaggcatatatcgctaaaattagccatcagaaccgttattgttattatattttcattacgaaagaggagagggcccagcgcgccagagcacacacggtcattgattactttatttggctaaagatccatcccttctcgatgtcatctctttccattcttgtgtatttttgattgaaaatgattttttgtccactaatttctaaaaataagacaaaaagcctttaagcagtttttcatccattttactacggtaaaatgaattagtacggtatggctcccagtcgcattatttttagattggccgtaggggctggggtagaactagagtaaggaacattgctctgccctcttttgaactgtcatataaatacctgacctattttattctccattatcgtattatctcacctctctttttctattctcttgtaattattgatttatagtcgtaactacaaagacaagcaaaataaaatacgttcgctctattaag

SEQ ID NO: 4 - DNA, 1014 bp, Saccharomyces cerevisiae
  CDS (1)..(1014): SED1 anchor
aaattatcaactgtcctattatctgccggtttagcctcgactactttggcccaattttccaacagtacatctgcttcttccaccgatgtcacttcctcctcttccatctccacttcctctggctcagtaactatcacatcttctgaagctccagaatccgacaacggtaccagcacagctgcaccaactgaaacctcaacagaggctccaaccactgctatcccaactaacggtacctctactgaagctccaaccactgctatcccaactaacggtacctctactgaagctccaactgatactactactgaagctccaaccaccgctcttccaactaacggtacttctactgaagctccaactgatactactactgaagctccaaccaccggtcttccaaccaacggtaccacttcagctttcccaccaactacatctttgccaccaagcaacactaccaccactcctccttacaacccatctactgactacaccactgactacactgtagtcactgaatatactacttactgtccagaaccaaccactttcaccacaaacggtaagacttacaccgtcactgaaccaaccacattgactatcactgactgtccatgcaccattgaaaagccaacaaccacatcaaccaccgaatacactgtagtcactgagtacactacttactgtccagaaccaaccactttcaccacaaacggtaagacttacaccgtcactgaaccaaccactttgactatcactgactgtccatgtactattgaaaagagcgaagcccctgagtcttctgtcccagttaccgaatctaagggcactaccaccaaagaaacaggtgttactaccaaacaaaccacagccaacccaagtctaaccgtctccacagtcgtcccagtttcatcctctgcttcttctcattccgttgtcatcaacagtaacggtgctaacgtcgtcgttccaggtgctttaggtttggctggtgttgctatgttattcttataa

SEQ ID NO: 5 - protein, 337 aa, Saccharomyces cerevisiae
KLSTVLLSAGLASTTLAQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 6 - DNA, 75 bp, Rhizopus oryzae
  CDS (1)..(75): Glucoamylase secretory signal
atgcaactgttcaatttgccattgaaagtttcattctttctcgtcctctcttacttttctttgctcgtttctgcc

SEQ ID NO: 7 - protein, 25 aa, Rhizopus oryzae
MQLFNLPLKVSFFLVLSYFSLLVSA

SEQ ID NO: 8 - DNA, 255 bp, Saccharomyces cerevisiae
  CDS (1)..(255): MF alpha prepro
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaaga

SEQ ID NO: 9 - protein, 85 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVSLDKR

SEQ ID NO: 10 - DNA, 300 bp, Saccharomyces cerevisiae
  CDS (1)..(300): HKR1 secretory signal
atggtctcattgaaaataaaaaaaattttactcctggtgtcattgttaaatgcaatcgaggcctatagtaacgatacaatatattcaacttcatacaataatggaatagaaagcacaccctcatattcaacatccgcgatatccagtaccggatctagcaacaaagagaatgcaataacatcaagctctgaaaccaccacaatggctggccaatatggtgaaagtggaagcacaacaataatggatgaacaagaaactggtacgtccagccagtatattagtgtgacgacgacaacgcaa

SEQ ID NO: 11 - protein, 100 aa, Saccharomyces cerevisiae
MVSLKIKKILLLVSLLNAIEAYSNDTIYSTSYNNGIESTPSYSTSAISSTGSSNKENAITSSSETTTMAGQYGESGSTTIMDEQETGTSSQYISVTTTTQ

SEQ ID NO: 12 - DNA, 1048 bp, Saccharomyces cerevisiae
  promoter (1)..(1048): TDH3 promoter
accagttctcacacggaacaccactaatggacacaaattcgaaatactttgaccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaaca

SEQ ID NO: 13 - DNA, 658 bp, Saccharomyces cerevisiae
  promoter (1)..(658): PGK1 promoter
aaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 14 - DNA, 2523 bp, Aspergillus aculeatus
  CDS (1)..(2523): beta-glucosidase
gatgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggaattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaagcttctgaaggccgagctcggcttccagggctttgtgatgtctgactggggtgctcaccacagtggtgttggctctgctttggccggcttggatatgtcaatgcctggcgatatcaccttcgattctgccactagtttctggggtaccaacctgaccattgctgtgctcaacggtaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggatccaactcgtacggtgccaatggctgctctgaccgtggctgtgacaacggtactcttgctatggcttggggtagcggcactgccgaattcccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccgggcgccaaatctccattcacctggggcaagacgagggaggcgtacggggattaccttgtccgtgagctcaacaacggcaacggagctccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaa

SEQ ID NO: 15 - protein, 841 aa, Aspergillus aculeatus
DELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 16 - DNA, 1191 bp, Trichoderma reesei
  CDS (1)..(1191): Endoglucanase II
cagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgacgacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggacttagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactggcagtggtaactcatggacggacacatccttggtcagctcgtgtctcgcaagaaag

SEQ ID NO: 17 - protein, 397 aa, Trichoderma reesei
QQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDLDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 18 - DNA, 516 bp, Saccharomyces cerevisiae
  promoter (1)..(516): CWP2 promoter
cgaactgaactgccttagctccgaagggcaattccacaggcactccgcggggcccggccaaggcccaaaaggcgtggaatatgcgcgttttggggccataacacccagtaccacggccggaacgggccatataataagtttttcactctcaagaatggtaaacgtaaataggaacatcccactaccctagaaattgcggaaatttcgcgcttatcattagaaaatctggaaccgtcctttttcctctttcttgcatttccctttccgtattattgccattctttaactgcatttggggaaccgtagaccaaaagccaaacagagaaatgtaacgttctaaaaaaaaaacaacgaaaaaattgaaaaataagatacaataatcgtatataaatcaggcttcttgttcatcattttcaattctcttcttgccatcccttttcctatctttgttcttttcttctcataatcaagaataaataacttcatcacattcgctacacactaacaagaaaaaaa

SEQ ID NO: 19 - DNA, 60 bp, Saccharomyces cerevisiae
  CDS (1)..(60): CWP2 secretory signal
atgcaattctctactgtcgcttccgttgctttcgtcgctttggctaactttgttgccgct

SEQ ID NO: 20 - protein, 20 aa, Saccharomyces cerevisiae
MQFSTVASVAFVALANFVAA

SEQ ID NO: 21 - DNA, 487 bp, Pichia pastoris
  promoter (1)..(487): TDH1 promoter
ttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 22 - DNA, 684 bp, Artificial Sequence
  Codon optimized mUkG1
atggtttccgttatcaaagaagagatgaagatcaagttgcacatggaaggtaacgttaacggtcacgctttcgttattgagggtgacggtaagggtaagccatacgacggtactcagactttgaacttgactgttaaggaaggtgctcctttgccattctcctacgacatcttgactaacgctttccagtacggtaacagagctttcactaagtacccagctgacatcccagactacttcaagcaaactttcccagagggttactcctgggagagaactatgtcttacgaggacaacgctatctgtaacgttagatccgaaatctctatggaaggtgactgtttcatctacaagatcagattcgacggtaagaacttcccaccaaacggtccagttatgcagaaaaagacattgaagtgggagccatccactgagatgatgtacgttagagatggattcttgatgggtgacgttaacatggctttgttgttggaaggtggtggtcaccacagatgtgacttcaagacttcctacaaggctaagaaggttgttcagttgccagacgctcacaagatcgaccacagaatcgaaattttgtcccacgacagagactactccaaggttaagttgtacgagaacgctgttgctagaaactccttgttgccatcccaggcttccaagtaa

SEQ ID NO: 23 - protein, 227 aa, Artificial Sequence
  Synthetic Construct
MVSVIKEEMKIKLHMEGNVNGHAFVIEGDGKGKPYDGTQTLNLTVKEGAPLPFSYDILTNAFQYGNRAFTKYPADIPDYFKQTFPEGYSWERTMSYEDNAICNVRSEISMEGDCFIYKIRFDGKNFPPNGPVMQKKTLKWEPSTEMMYVRDGFLMGDVNMALLLEGGGHHRCDFKTSYKAKKVVQLPDAHKIDHRIEILSHDRDYSKVKLYENAVARNSLLPSQASK

SEQ ID NO: 24 - DNA, 47 bp, Artificial Sequence
  Sed1p-F
cctcttcgctattacgccagattggatatagaaaattaacgtaaggc

SEQ ID NO: 25 - DNA, 40 bp, Artificial Sequence
  Sed1ss-R
ggagagaacgccagttcatcttgggccaaagtagtcgagg

SEQ ID NO: 26 - DNA, 42 bp, Artificial Sequence
  BGL1-F
cctcgactactttggcccaagatgaactggcgttctctcctc

SEQ ID NO: 27 - DNA, 40 bp, Artificial Sequence
  PRS-R
gttaattttctatatccaatctggcgtaatagcgaagagg

SEQ ID NO: 28 - DNA, 46 bp, Artificial Sequence
  MFa-F
aatacgttcgctctattaagatgagatttccttcaatttttactgc

SEQ ID NO: 29 - DNA, 34 bp, Artificial Sequence
  MFa-R
ggagagaacgccagttcatcgaattcgtcgacgt

SEQ ID NO: 30 - DNA, 42 bp, Artificial Sequence
  BGL1-F2
acgtcgacgtcgacgaattcgatgaactggcgttctctcctc

SEQ ID NO: 31 - DNA, 48 bp, Artificial Sequence
  Sed1p-R
aaaattgaaggaaatctcatcttaatagagcgaacgtattttattttg

SEQ ID NO: 32 - DNA, 40 bp, Artificial Sequence
  Hkr1ss-F
aatacgttcgctctattaagatggtctcattgaaaataaa

SEQ ID NO: 33 - DNA, 38 bp, Artificial Sequence
  Hkr1ss-R
ggagagaacgccagttcatcttgcgttgtcgtcgtcac

SEQ ID NO: 34 - DNA, 42 bp, Artificial Sequence
  BGL1-F3
gtgtgacgacgacaacgcaagatgaactggcgttctctcctc

SEQ ID NO: 35 - DNA, 48 bp, Artificial Sequence
  Sed1p-R2
tttattttcaatgagaccatcttaatagagcgaacgtattttattttg

SEQ ID NO: 36 - DNA, 34 bp, Artificial Sequence
  PRS-BsrGI-F
gccatctgtacagttagtacattgagtctaaata

SEQ ID NO: 37 - DNA, 31 bp, Artificial Sequence
  BGL1-BsrGI-R
gccatctgtacatcattgcaccttcgggagc

SEQ ID NO: 38 - DNA, 38 bp, Artificial Sequence
  EGII-F
cctcgactactttggcccaacagcagactgtctggggc

SEQ ID NO: 39 - DNA, 38 bp, Artificial Sequence
  EGII-R
aataggacagttgataatttactcgagccctttcttgc

SEQ ID NO: 40 - DNA, 47 bp, Artificial Sequence
  Sed1a-F
tcgcaagaaagggctcgagtaaattatcaactgtcctattatctgcc

SEQ ID NO: 41 - DNA, 40 bp, Artificial Sequence
  Sed1ss-R2
tggccccagacagtctgctgttgggccaaagtagtcgagg

SEQ ID NO: 42 - DNA, 38 bp, Artificial Sequence
  EGII-F2
acgtcgacgtcgacgaattccagcagactgtctggggc

SEQ ID NO: 43 - DNA, 34 bp, Artificial Sequence
  MFa-R2
tggccccagacagtctgctggaattcgtcgacgt

SEQ ID NO: 44 - DNA, 36 bp, Artificial Sequence
  TDH3p-F
cctcttcgctattacgccagatcgccaagcgcgcaa

SEQ ID NO: 45 - DNA, 50 bp, Artificial Sequence
  TDH3p-R
aggacagttgataatttcatgaattctgtttatgtgtgtttattcgaaac

SEQ ID NO: 46 - DNA, 50 bp, Artificial Sequence
  Sed1ss-F
aacacacataaacagaattcatgaaattatcaactgtcctattatctgcc

SEQ ID NO: 47 - DNA, 40 bp, Artificial Sequence
  PRS-R2
ttaattgcgcgcttggcgatctggcgtaatagcgaagagg

SEQ ID NO: 48 - DNA, 50 bp, Artificial Sequence
  TDH3p-R2
aaaattgaaggaaatctcatgaattctgtttatgtgtgtttattcgaaac

SEQ ID NO: 49 - DNA, 46 bp, Artificial Sequence
  MFa-F2
aacacacataaacagaattcatgagatttccttcaatttttactgc

SEQ ID NO: 50 - DNA, 38 bp, Artificial Sequence
  PGK1p-F
cctcttcgctattacgccagaaagatgccgatttgggc

SEQ ID NO: 51 - DNA, 54 bp, Artificial Sequence
  PGK1p-R
ggcaaattgaacagttgcattgttttatatttgttgtaaaaagtagataattac

SEQ ID NO: 52 - DNA, 44 bp, Artificial Sequence
  GAss-F
ttttacaacaaatataaaacaatgcaactgttcaatttgccatt

SEQ ID NO: 53 - DNA, 40 bp, Artificial Sequence
  PRS-R3
gcgcccaaatcggcatctttctggcgtaatagcgaagagg

SEQ ID NO: 54 - DNA, 54 bp, Artificial Sequence
  PGK1p-R2
aggacagttgataatttcattgttttatatttgttgtaaaaagtagataattac

SEQ ID NO: 55 - DNA, 51 bp, Artificial Sequence
  Sed1ss-F2
ttttacaacaaatataaaacaatgaaattatcaactgtcctattatctgcc

SEQ ID NO: 56 - DNA, 54 bp, Artificial Sequence
  PGK1p-R3
aaaattgaaggaaatctcattgttttatatttgttgtaaaaagtagataattac

SEQ ID NO: 57 - DNA, 47 bp, Artificial Sequence
  MFa-F3
ttttacaacaaatataaaacaatgagatttccttcaatttttactgc

SEQ ID NO: 58 - DNA, 42 bp, Artificial Sequence
  Cwp2p-F
cctcttcgctattacgccagcgaactgaactgccttagctcc

SEQ ID NO: 59 - DNA, 48 bp, Artificial Sequence
  Cwp2p-R
aggacagttgataatttcattttttttcttgttagtgtgtagcgaatg

SEQ ID NO: 60 - DNA, 50 bp, Artificial Sequence
  Sed1ss-F3
acacactaacaagaaaaaaaatgaaattatcaactgtcctattatctgcc

SEQ ID NO: 61 - DNA, 40 bp, Artificial Sequence
  PRS-R4
agctaaggcagttcagttcgctggcgtaatagcgaagagg

SEQ ID NO: 62 - DNA, 42 bp, Artificial Sequence
  Cwp2ss-R
ggagagaacgccagttcatcagcggcaacaaagttagccaaa

SEQ ID NO: 63 - DNA, 42 bp, Artificial Sequence
  BGL1-F4
tggctaactttgttgccgctgatgaactggcgttctctcctc

SEQ ID NO: 64 - DNA, 39 bp, Artificial Sequence
  G418r-BamHI-F
cgacggccagtgaattcttagaaaaactcatcgagcatc

SEQ ID NO: 65 - DNA, 38 bp, Artificial Sequence
  G418r-EcoRI-R
tcgactctagaggatccgctttagttgggcataatctg

SEQ ID NO: 66 - DNA, 49 bp, Artificial Sequence
  MFa-SpeI-F
gaacaactatcaaaacacaactagtatgcaactgttcaatttgccattg

SEQ ID NO: 67 - DNA, 43 bp, Artificial Sequence
  MFa-XhoI-R
ttgataacggaaaccatctcgagcgcggcagaaacgagcaaag

SEQ ID NO: 68 - DNA, 49 bp, Artificial Sequence
  Sed1ss-SpeI-F
acaactatcaaaacacaactagtatgaaattatcaactgtcctattatc

SEQ ID NO: 69 - DNA, 42 bp, Artificial Sequence
  Sed1ss-XhoI-R
tgataacggaaaccatctcgagttgggccaaagtagtcgagg